(19) United States
(12) Patent Application Publication
(10) Pub. No.: US 2010/0144022 A1
(43) Pub. Date: Jun. 10, 2010

(54) Continuous Flow Bioreactor

(76) Inventor: Krishna P. Surapaneni, Anaheim Hills, CA (US)

Correspondence Address: PATENTMETRIX, 14252 CULVER DR. BOX 914, IRVINE, CA, 92604, US

(21) Appl. No.: 12/610,340

(22) Filed: Nov. 1, 2009

Related U.S. Application Data
(60) Provisional application No. 61/113,557, filed on Nov. 11, 2008.
(60) Provisional application No. 61/223,061, filed on Jul. 5, 2009.

(51) Int. Cl. C12M 3/00; C12M 1/00

(52) U.S. Cl. 435/289.1

(57) ABSTRACT

The present invention is directed toward bioreactor systems capable of causing internal contents to have an angular motion throughout the bioreactor container without rotating the container. In one embodiment, the bioreactor system has a base platform, including three areas, a toroidal container removably attached to the base platform, a three separate members physically attached to the three areas, and a plurality of motors, where each of the motors is configured to cause an independent translational movement in each of the members and where the translation movement in each of the members causes a corresponding translational movement in a portion of the toroidal container proximate to the moving area.

### CROSS REFERENCE

This application relies on U.S. Provisional Application No. 61/113,557, filed on Nov. 11, 2008, and U.S. Provisional Application No. 61/223,061, filed on Jul. 5, 2009, for priority. Both applications are herein incorporated by reference.

### FIELD OF THE INVENTION

The present invention relates to culture systems for culturing or growing bacterial, fungal, mammalian, insect, viral, plant or any other living cell types. In particular, the present invention is a system and method for culturing cells for a variety of uses by employing a hollow, ring-shaped or annular container which when, agitated by external means, such as a platform, effectuates a circular motion of the contents contained within the annular container, thereby improving yields of desirable cell culture products.

### BACKGROUND OF THE INVENTION

Traditionally, cells are cultivated in small, medium and large scale in bioreactors or “fermentors”. The fermentors typically have rigid glass or stainless steel vessels, for containing the cells, which are connected to an impeller or other agitation mechanism for stirring the contents of the vessels. The vessels are typically cylindrical in shape, having either a round or flat bottom. The agitation mechanism is actuated by mechanical means, electrical means, or aeration.

Conventional bioreactor systems use either internal or external agitation mechanisms to maintain cells under suspension. Internal stir mechanisms, such as impellers which are typically used to stir the contents of the vessels, are of different shapes and types and stirring speeds range from a few RPH to hundreds of RPM. Current internal stirring mechanisms for cultivation of cell cultures are sub-optimal in that the stirring is disruptive and is likely to harm (i.e. break) the cells under suspension. In the case of cells growing on microcarriers, the stirring action may produce large shear forces that effectively dislodge the cells from the microcarriers and potentially cause cell lysis.

External agitation mechanisms include agitation by means of a rocking platform, such as the wave bioreactor described in U.S. Pat. No. 6,544,788, assigned to GE Healthcare Bioscience Bioprocess Group and herein incorporated by reference, which describes “[a] bioreactor assembly comprising: a chamber capable of receiving a liquid media; and a filter disposed in the chamber, the filter being free to move within the chamber, further comprising: a rocking platform on which the chamber is located, whereby rocking of the rocking platform induces the wave motion in the liquid media received in the chamber.” Conventional bioreactor systems that culture cells using external stirring mechanisms or air lift may be exposed to collisions among the suspended cells. In addition, there is the possibility of the formation of turbulence that may result in inefficient stirring. Furthermore, in conventional bioreactors, there are invariably areas located on the lowest parts of the reaction vessel (dependent areas) where sedimentation might occur and the stirring mechanism could fail to keep the cells in suspension, resulting in potential detriment to the cells and thus, poor yields.

U.S. Pat. No. 6,190,913 to Singh, which is incorporated herein by reference, describes “[a] method for the cultivation of cells comprising the steps of providing a pre-sterilized plastic bag having a volume of at least five liters, the bag having a single hollow interior chamber; partially filling the bag with a gas containing oxygen to thereby partially inflate the bag; introducing a liquid media and a cell culture into the bag, wherein the liquid media and the cell culture comprise between 10 to 80% of the volume of the bag; filling the remainder of the bag with the gas such that the bag becomes rigid; securing the bag to a platform; rocking the platform in a single degree of freedom to thereby induce a wave motion to the liquid media in the bag, whereby the necessary oxygen transfer and mixing required for cell growth and productivity is accomplished by the wave motion.”

United States Patent Application No. 20080160597, assigned to Cellution Biotech B. V., which is herein incorporated by reference, describes “[a] method for cultivating cells utilizing wave motion, comprising the steps of: providing a container; introducing a gas containing oxygen, a liquid medium and a cell culture into the container; moving the container such that the container swivels with respect to a substantially horizontal pivot axis to thereby induce a wave motion to the liquid medium in the container, which wave motion contributes to the necessary oxygen transfer and mixing required for cell growth, characterized in that during said swivelling of the container said pivot axis follows a cyclical closed-loop path.”

Conventional agitation mechanisms are also disadvantageous in that they pose problems of aeration efficiency and of keeping the cells in proper suspension, especially with larger cell types, such as animal cell cultures, since the cells tend to either settle down or collide with the impeller or other cells.

Further, conventional designs employ steam for in situ cleaning and sterilization, which is not only time-consuming, laborious, and expensive, but requires proper validation. Recently, disposable bioreactors have been developed, formed from flexible plastic material that is pre-sterilized using radiation. The disposable bioreactors thus obviate the necessity for cleaning and in situ sterilization because they are only used once and discarded.

Still further, conventional bioreactors are also used as perfusion systems whereby the liquid from the bioreactors is pumped out using a filter so that cultured cells are retained in the container. Conventional perfusion methods are disadvantageous in that they tend to clog filters since the filtration is essentially of the cross flow type.

Thus, the prior art rigid and disposable bioreactor systems, and methods for using such systems, are disadvantageous for the several reasons also described above.

What is therefore needed is a system and method for cultivating cells that improves usable cell yield by reducing sedimentation, reducing cell breakage, improving aeration efficiency, and improved stirring, among other benefits.

### SUMMARY OF THE INVENTION

In one embodiment, the present invention is directed toward a bioreactor system comprising a base platform having a flat surface, a first area, a second area, and a third area, wherein each of said first, second, and third areas are separated by a distance; a toroidal container removably attached to said base platform; a first member physically attached to said first area; a second member physically attached to said second area; a third member physically attached to said third area; a plurality of motors, wherein each of said motors is configured to cause an independent translational movement in said first member, said second member, and said third member, and wherein said translation movement in each of said first member, second member, and third member causes a corresponding translational movement in a portion of said toroidal container proximate to said first area, said second area, and said third area, respectively.

The translational movement is either upward or downward. The bioreactor system further comprises a controller for controlling a sequence of said translational movement. Optionally, the sequence comprises causing an upward translational movement of said first area substantially concurrently with causing a downward translational movement of said second area. Optionally, the sequence further comprises, after said causing an upward translational movement of said first area substantially concurrently with causing a downward translational movement of said second area, causing an upward translational movement of said second area substantially concurrently with causing a downward translational movement of said first area. The sequence causes contents within said toroidal container to have an angular motion throughout said toroidal container without requiring a physical rotation of said container.

Optionally, the plurality of motors comprises a first motor, a second motor and a third motor, wherein said first motor is configured to cause a translational movement in said first member, said second motor is configured to cause a translational movement in said second member, and said third motor is configured to cause a translational movement is said third member. Optionally, the platform rests atop a support member comprising a pivot point. The pivot point comprises a spherical surface to which said a bottom surface of said platform is attached. The toroidal container has a first internal volume and a second internal volume, wherein said second internal volume is defined by a second toroidal container housed within said toroidal container. The second toroidal container comprises a filter. The second toroidal container comprises an input port and/or output port. The toroidal container comprises an output port and/or an input port.

In another embodiment, the bioreactor system comprises a base platform having a flat surface, a first area, a second area, and a third area, wherein each of said first, second, and third areas are separated by a distance; a toroidal container removably attached to said base platform; a first member physically attached to said first area; a second member physically attached to said second area; a third member physically attached to said third area; a first motor, wherein said first motor is configured to cause a first translational movement in said first member; a second motor, wherein said second motor is configured to cause a second translational movement in said second member; and a third motor, wherein said third motor is configured to cause a third translational movement in said third member, wherein each of said first, second, and third translational movements are capable of occurring independent of each other and wherein said first, second, and third translation movements by each of said first, second, and third members causes a corresponding translational movement in a portion of said toroidal container proximate to said first area, said second area, and said third area, respectively.

Optionally, the bioreactor system further comprises a controller for controlling a sequence of said translational movement. The sequence comprises causing an upward translational movement of said first area substantially concurrently with causing a downward translational movement of said second area. The sequence further comprises, after said causing an upward translational movement of said first area substantially concurrently with causing a downward translational movement of said second area, causing an upward translational movement of said second area substantially concurrently with causing a downward translational movement of said first area. The sequence causes contents within said toroidal container to have an angular motion throughout said toroidal container without requiring a physical rotation of said container. The toroidal container has a first internal volume and a second internal volume, wherein said second internal volume is defined by a second toroidal container housed within said toroidal container. The bioreactor second toroidal container comprises a filter.

These and other embodiments will become fully understood in the Detailed Description with reference to the drawings.

### BRIEF DESCRIPTION OF THE DRAWINGS

These and other features and advantages of the present invention will be appreciated, as they become better understood by reference to the following detailed description when considered in connection with the accompanying drawings, wherein:

FIG. 1 illustrates a first embodiment of the continuous flow bioreactor of the present invention;

FIG. 2 is a side view of a first embodiment of the continuous flow bioreactor of the present invention;

FIG. 3 depicts a top perspective view of a first embodiment of the continuous flow bioreactor of the present invention, delineating cross sections shown in further detail in FIGS. 4a, 4b, and 4c;

FIG. 4a is a first sectional side view of one embodiment of the continuous flow bioreactor of the present invention, in a first stage of agitation;

FIG. 4b is a second sectional side view of one embodiment of the continuous flow bioreactor of the present invention, in a second stage of agitation;

FIG. 4c is a third sectional side view of one embodiment of the continuous flow bioreactor of the present invention, in a third stage of agitation;

FIG. 4d is an illustration of one embodiment of the bioreactor of the present invention in which at least one filter is optionally placed at the top or bottom of the container;

FIG. 4e depicts another embodiment of the bioreactor of the present invention, illustrating a container-within-container perfusion bioreactor;

FIG. 4f depicts a section of another embodiment of the bioreactor of the present invention, illustrating a container-within-container perfusion bioreactor;

FIG. 5 is a top perspective view of a second embodiment of the continuous flow bioreactor of the present invention;

FIG. 5a is a top perspective view of one embodiment of the continuous flow bioreactor of the present invention in a first stage of agitation;

FIG. 5b is a top perspective view of one embodiment of the continuous flow bioreactor of the present invention in a second stage of agitation;

FIG. 5c is a top perspective view of one embodiment of the continuous flow bioreactor of the present invention in a third stage of agitation;

FIG. 5d is a top perspective view of one embodiment of the continuous flow bioreactor of the present invention in a fourth stage of agitation;

FIG. 6 is a top perspective view of another embodiment of the continuous flow bioreactor of the present invention;

FIG. 6a is a top perspective views of another embodiment of the continuous flow bioreactor of the present invention in a first stage of agitation;

FIG. 6b is a top perspective views of another embodiment of the continuous flow bioreactor of the present invention in a second stage of agitation;

FIG. 6c is a top perspective views of another embodiment of the continuous flow bioreactor of the present invention in a third stage of agitation;

FIG. 6d is a top perspective views of another embodiment of the continuous flow bioreactor of the present invention in a fourth stage of agitation;

FIG. 7a is perspective view of another embodiment of the continuous flow bioreactor of present invention, in tube in tube configuration, in a first stage of agitation;

FIG. 7b is perspective view of another embodiment of the continuous flow bioreactor of present invention, in tube in tube configuration, in a second stage of agitation;

FIG. 7c is perspective view of another embodiment of the continuous flow bioreactor of present invention, in tube in tube configuration, in a third stage of agitation;

FIG. 7d is perspective view of another embodiment of the continuous flow bioreactor of present invention, in tube in tube configuration, in a fourth stage of agitation;

FIG. 8a is a perspective view of another embodiment of continuous flow bioreactor in a stacked configuration;

FIG. 8b is a perspective view of another embodiment of continuous flow bioreactor in a stacked configuration; and

FIG. 9 is a perspective view of another embodiment of the continuous flow bioreactor of the present invention, as shown in FIG. 6 in a hub and rim configuration, in a stacked configuration.

### DETAILED DESCRIPTION

While the present invention may be embodied in many different forms, for the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

“Duration” and variations thereof refer to the time course of a prescribed treatment, from initiation to conclusion, whether the treatment is concluded because the condition is resolved or the treatment is suspended for any reason. Over the duration of treatment, a plurality of treatment periods may be prescribed during which one or more prescribed stimuli are administered to the system.

The term “and/or” means one or all of the listed elements or a combination of any two or more of the listed elements.

The terms “comprises” and variations thereof do not have a limiting meaning where these terms appear in the description and claims.

Unless otherwise specified, “a,” “an,” “the,” “one or more,” and “at least one” are used interchangeably and mean one or more than one.

For any method disclosed herein that includes discrete steps, the steps may be conducted in any feasible order. And, as appropriate, any combination of two or more steps may be conducted simultaneously.

Also herein, the recitations of numerical ranges by endpoints include all numbers subsumed within that range (e.g., 1 to 5 includes 1, 1.5, 2, 2.75, 3, 3.80, 4, 5, etc.). Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The present invention is directed towards culture systems for culturing or growing bacterial, fungal, mammalian, insect, viral, plant or any other living cell types used for, among other uses, studies related to growth and other characteristics, for production of metabolites, antibodies, proteins, viruses, polysaccharides or appropriate components and derivative of such cells in culture in closed or semi-closed reactor systems, in batch systems, or fed batch systems or continuous culture systems, including perfusion systems. The applications include and are not limited to industrial, research & development, production, medical, organ transplants and synthetic organs.

Specifically, the present invention is, in one embodiment, directed toward a system and method for cultivating cells that improves usable cell yield by employing a hollow, annular (ring-shaped) or toroidal container in conjunction with an external agitation mechanism of the container, which, in one embodiment, employs a non-rotating, circular, swaying motion. In one embodiment, the agitation of the container results in a circular motion of the contents contained within the annular container, improving yields of desirable cell culture products.

More specifically, the present invention provides a method for cultivating cells wherein the method comprises cultivating cells under a gentle sway motion that is substantially devoid of wave motion or severe agitation. In one embodiment, the gentle sway motion represents subjecting the cells to motions ranging from 5 motions per minute to 60 motions per minute. In one embodiment, the gentle sway motion represents subjecting the cells to motions less than 60 motions per minute.

In another embodiment, the gentle sway motion may be circular or substantially circular, elliptical, or any other geometric shape so long as the geometric shape does not provide an acute angle of less than 25 degrees. It is understood that such acute angle may be not conducive to optimal exposure of the cells to the growth medium. As stated herein, the phrase “devoid of wave motion or severe agitation” means a gentle sway motion that does not produce distinct wave-shaped fluid motion in the culture medium fluid. In one aspect, the gentle sway motion does not produce a wave motion where the height of what waves are produced is no more than three times the base of the wave.

In another embodiment, the gentle sway motion may not be continuous as the cells may be subjected to gentle rocking motion such that it does not cause the cells present in one area of the container to relocate to a distant area of the container.

More specifically, in one embodiment, the present invention is directed towards a continuous flow bioreactor having a container, where in one embodiment, the container is in the shape of toroid, with or without a central hole, having an annular chamber to house contents. In one embodiment, the container is rigid and formed from an appropriate material such as stainless steel or glass. In another embodiment, the container is disposable and is formed from an appropriate material such as a flexible plastic (i.e disposable bag) that can be used for a variety of cell cultures.

In another embodiment, the container further includes an inlet port for gas to help with agitation. In one embodiment, the inlet is designed for use with an inert gas.

Still more specifically, in one embodiment, the present invention is directed towards a continuous flow bioreactor having a container resting on a platform, either alone or mounted on a rim.

In a first embodiment, the external agitation mechanism moves a connector rod, in three axes or planes, causing movement of the platform and container and thus, fluid within the container. In one embodiment, the agitation mechanism is programmable to control speed, number of rotations, and swaying angles.

In a second embodiment, the external agitation mechanism moves the platform, and thus container resting on platform, via a top surface agitation method. In one embodiment, the platform comprises a rigid material capable of withstanding weight in the range of 1000 to 3000 kilograms. The platform, in one embodiment, is connected to a motor and levers in all three planes or axes of movement, thus creating a pitch, yaw, and roll movement. The motors are programmable to create a swaying motion of the platform and container, which in turn, creates smooth circular motion of the fluid within the container. In one embodiment, the platform rests on a central pivoting arm or rod. In one embodiment, the platform further comprises an expandable container holder. The expandable container holder facilitates the use of different sizes of flexible or rigid containers, wherein the containers house the cells that can be harvested.

In a third embodiment, an external agitation mechanism moves a hub connected through the center of a rim upon which the container is attached, thus moving the rim. In one embodiment, the rim is removably connected to the platform. When rotated via an external agitation mechanism that is connected to a hub on the rim, the entire assembly sways from one point to the other, thus mixing the contents of the container in circumferential motion. Further, the hub and rim configuration, in one embodiment, comprises an expandable container holder made of a rigid material that facilitates the use of different sizes of flexible or rigid containers, and thus cell volumes. In one embodiment, the expandable container holder rests on a spherical or rectangular rim which is connected to the central hub by radiating spokes. The central hub is connected to a lever, which in turn, is connected to a motor. In one embodiment, the motor is programmable to allow for manipulation of rotational speed. The agitation lever can be mounted either at the top of the hub for top down agitation or the bottom of the hub for bottom up agitation.

In all embodiments, it should be noted that the speed of agitation can be controlled and depends upon working load and density and viscosity of the medium/media present in the bioreactor.

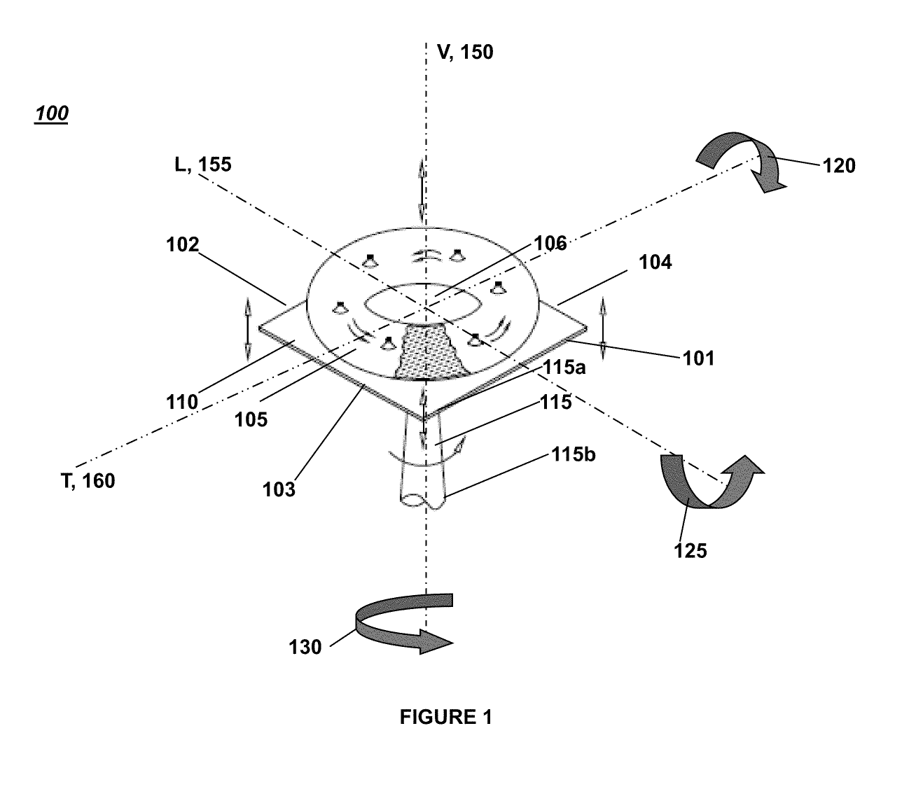

FIG. 1 is a top perspective view of a first embodiment of the continuous flow bioreactor 100 of the present invention. The continuous flow bioreactor 100 comprises a container 105. In one embodiment, the container 105 is in the shape of a toroid. In one embodiment, the walls of container 105 form a central opening 106 so that the container 105 is an annular chamber capable of housing contents such as cell cultures.

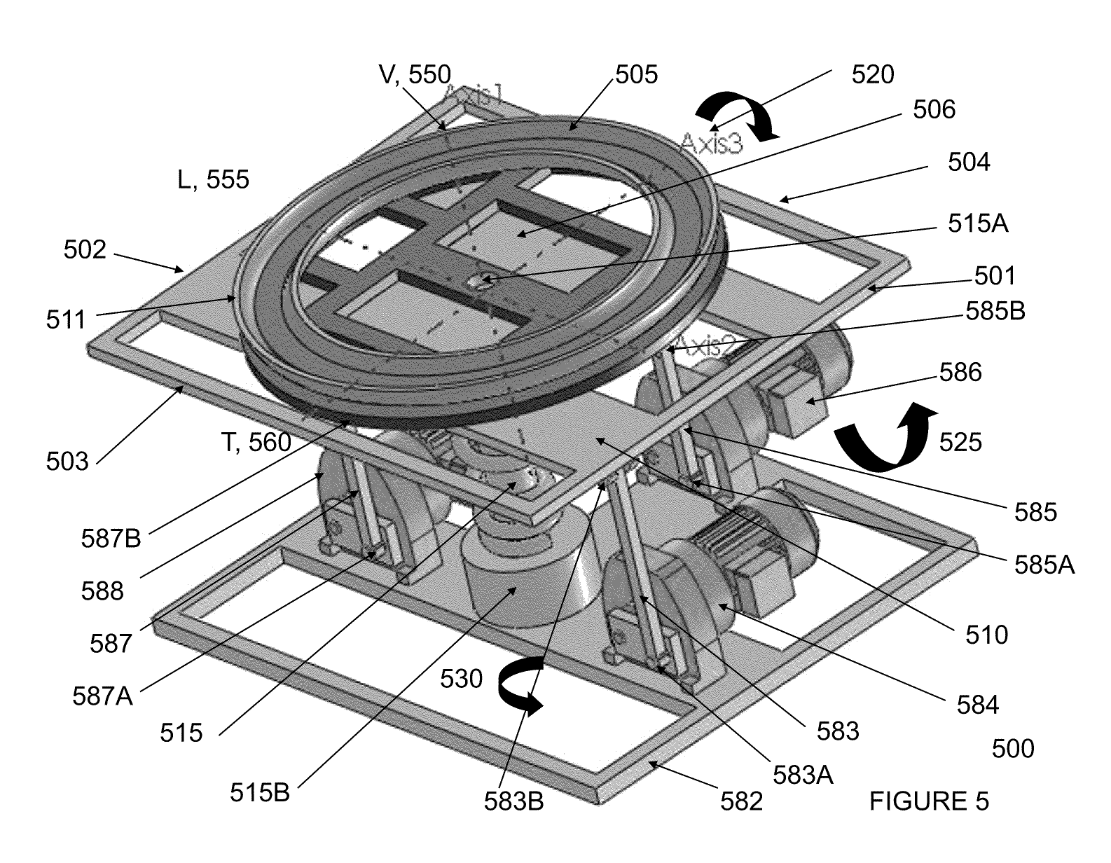

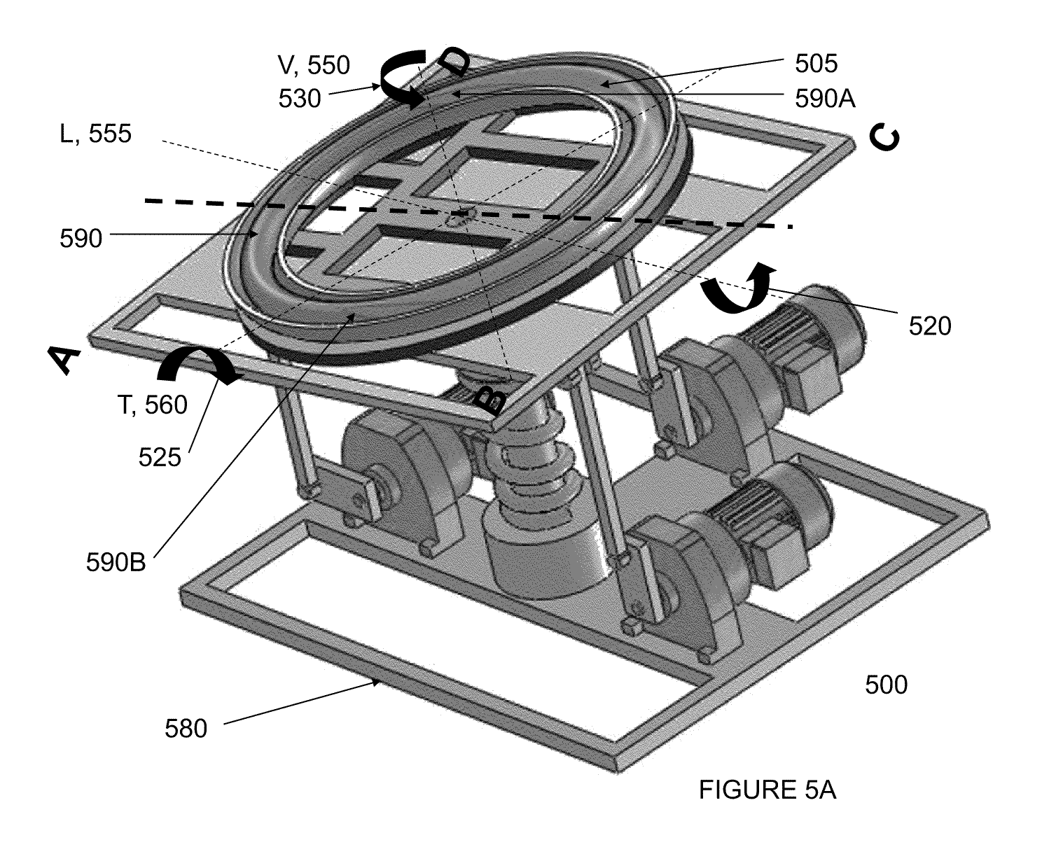

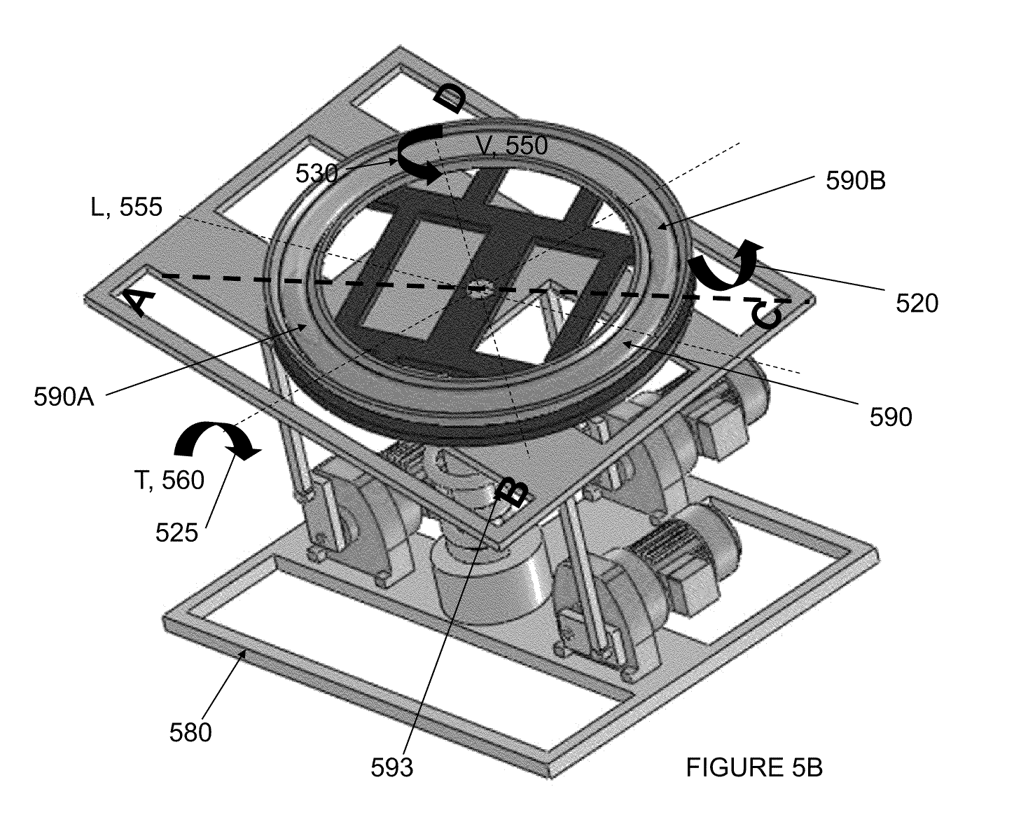

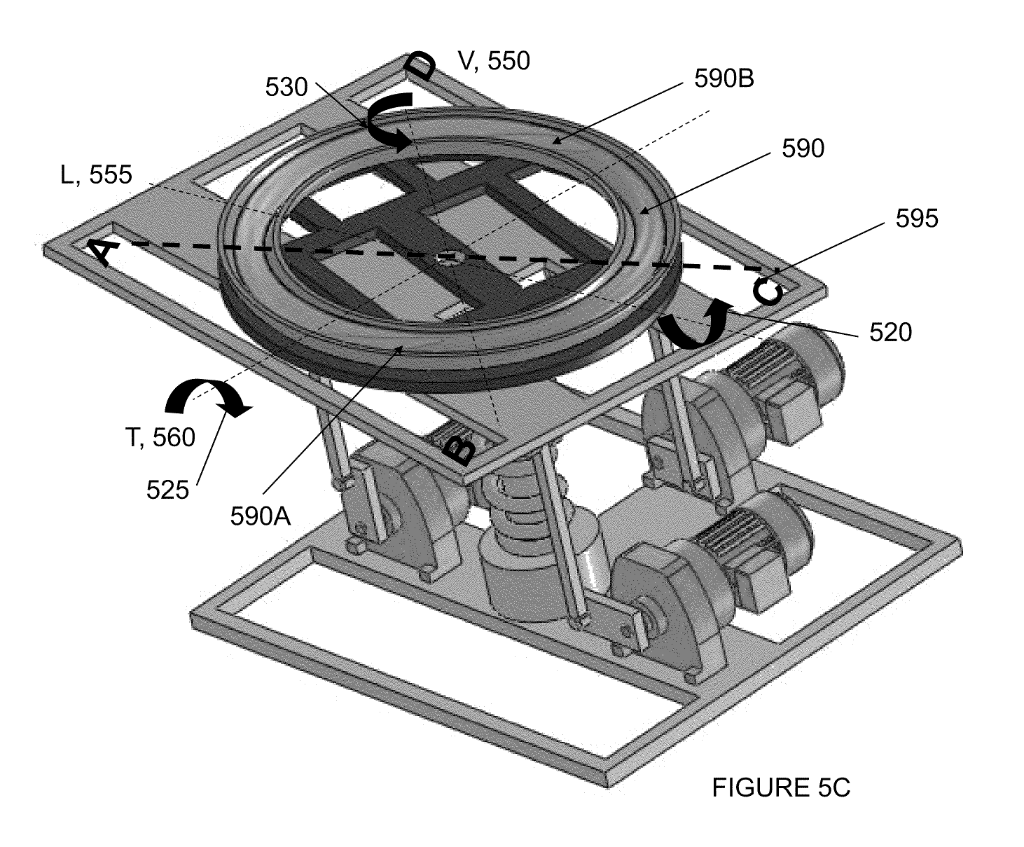

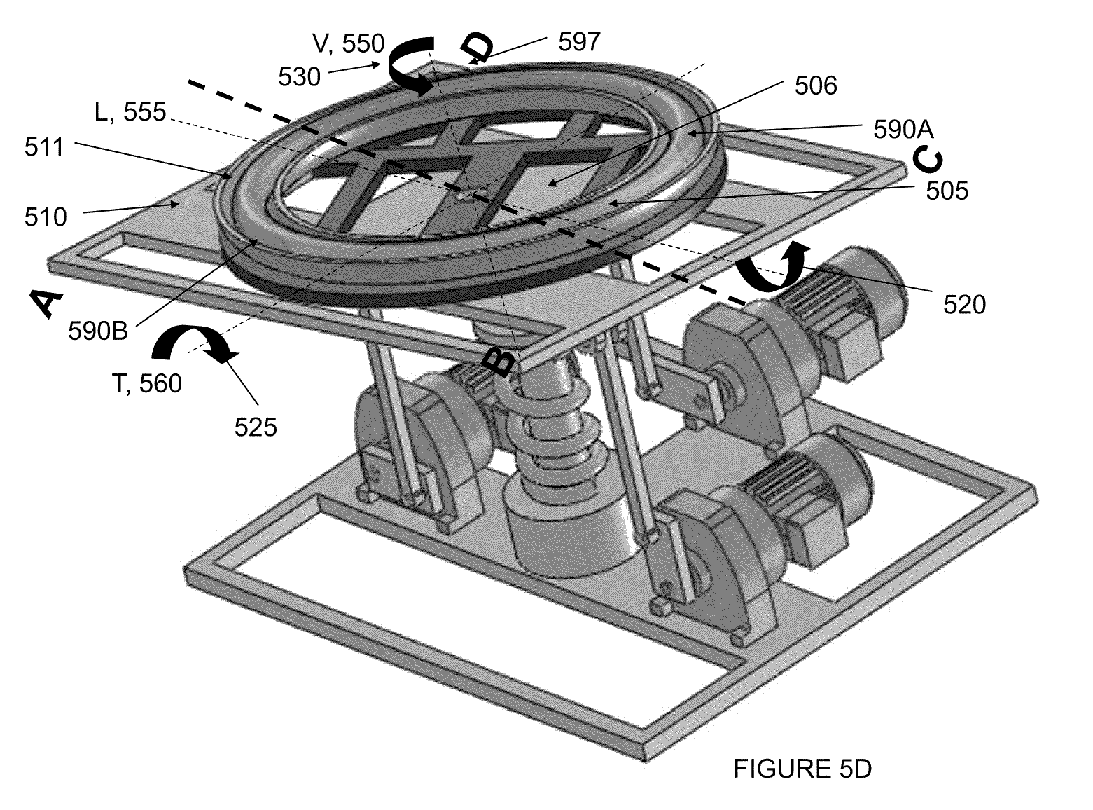

In one embodiment, the container 105 is rigid and formed from an appropriate material such as stainless steel or glass. In another embodiment, the container 105 is disposable and is formed from an appropriate material such as a flexible plastic (disposable bag) that is inflatable using a filtered air stream and can be used for a variety of cell cultures. The continuous flow bioreactor of the present invention further comprises a platform 110, upon which an expandable container holder (shown in FIG. 5 and described in greater detail with respect to FIG. 5) rests. In one embodiment, container 105 rests in the expandable container holder.

Platform 110 may be of any shape, but is preferably square, circular or rectangular. Platform 110 is movably attached to connector cone 115 at the cone's pointed, proximal end 115a. At its distal end 115b, connector cone 115 is positioned on a stationary base, which is a solid, stable surface. Programmable motors are connected to the platform 110 via linkages in all three axes of motion. It should be appreciated that any agitation mechanism, known to persons of ordinary skill in the art, that can provide a substantially rocking movement to the container 105 can be used, including a rocking platform, a gyrating base, or any other means to provide the kind of motion described herein. In one embodiment, connector cone is further equipped with a spherical structure or knob at its pointed, proximal end 115a to enable smoother agitation.

The motion of the platform 110 and hence the resulting circumferential motion of the contents of container 105, therefrom, is understood with reference to a set of axes—a) vertical axis ‘V’ 150 that passes through the center of the platform 110 and along which resides the connector cone 115 in a parallel orientation; b) longitudinal axis ‘L’ 155 and c) transverse axis ‘T’ 160. It should be appreciated that, to agitate the platform 110, a plurality of motors connected to various points on to said platform via pulleys or other attachment mechanisms may be used to effectuate the movement detailed below and therefore function as the agitation mechanism. Further, the motors can be controlled via a controller and/or processor, which is wired or wirelessly connected to the motors, that executes controls or instructions stored in local memory. The controls or instructions are input by a user via an input means, including a display, keyboard, mouse, touchscreen, or other input mechanism. Once input, the instructions, such as the time for agitation, degree of agitation, intensity of agitation, among other variables, are used to drive, control, or otherwise operate the motors and, therefore, the platform agitator.

In one embodiment, the agitation mechanism effectuates two repeated sequential motions of the platform 110 and the container 105: a first rolling motion 125 around the longitudinal axis ‘L’ and a second pitching motion 120 around the transverse axis ‘T’. For example, the platform 110 may first have angular motion 120 followed by the angular motion 125, or vice versa, repeated one after another in a continuous loop for a specified duration of time, as determined by the input instructions described above. In other words, motion 120 causes platform side 101 to move downwards while the opposite side 102 moves up. This is followed by motion 125 that next causes, for example, side 103 to move downwards while side 104 moves up. Next, motion 120 causes side 102 to move down while the opposite side 101 moves up; followed by motion 125 that causes side 104 to move down while side 103 moves up. This entire cycle is repeated continuously. It should be noted that the motions 120, 125 do not cause the platform 110 or the container 105 to rotate around the vertical axis ‘V’ 150.

An external agitation mechanism (not shown) provides the two angular motions to the platform via connecting cone 115. In one embodiment, a spherical knob at pointed, proximal end 115a enables smoother motion of the platform 110 and hence of the reactor 105 and its contents. The continuous and repeated combination of the two angular motions 120, 125 results into a continuous, side-to-side as well as circumferential motion 130 of the contents of the container 105, around vertical axis ‘V’ 150, without actually rotating the entire platform 110 and reactor 105. Thus, the contents of the container 105 are agitated with a combination circumferential motion 130 around vertical axis ‘V’ 150 and a side-to-side swaying against walls of the container.

Persons of ordinary skill in the art should appreciate that the rolling and pitching motions 125, 120 of the platform 110 generate upward and downward acceleration forces directed tangentially to the direction of angular motions, the values of which increase with distance from the rolling or pitching axis (that is, axis L 155 and T 160 respectively) and are inversely proportional to the square of the rolling or pitching periods. In one embodiment, the acceleration forces that resultantly act on the container 105 and hence on the contents therein can be controlled by any one or combination of at least the following factors: a) average radius of the container 105; b) rolling and pitching motion time periods; and c) rolling and pitching motion angles. Thus, at an identical distance from the axes L and T, if the rolling or pitching period is halved, acceleration forces acting on the container 105 are quadrupled, while if the rolling or pitching period is doubled, acceleration forces are quartered. Rolling or pitching motion tilt-angles generate down-slope forces on the container 105 that rests on the platform 110. In one embodiment, the tilt-angles for motions 120, 125 are kept in the range of 0-45 degrees to prevent slippage of the container 105 over platform 110 due to the down-slope forces. With the use of appropriate fixtures or stops on the platform 110 to hold the container 105 thereon, the tilt-angles for motions 120, 125 can be steeper and in the range of 0-45 degrees without causing the container 105 to topple over.

Also, in one embodiment, the speed of the angular motions 120, 125 depends upon at least the working load of the container 105 and the density and viscosity of the medium/media present in the bioreactor.

It should be appreciated that the above described variables can be controlled through the aforementioned input instructions. More specifically, if greater agitation is required, then, relative to the average radius of the container being used (which is preferably a variable that can be input as an instruction), the rolling and pitching motion time periods are increased. If less agitation is required, then, relative to the average radius of the container being used, the rolling and pitching motion time periods are decreased. If greater agitation is required, then, relative to the average radius of the container being used, the rolling and pitching motion time angles are increased, that is approaching the 45 degree angle described above. If less agitation is required, then, relative to the average radius of the container being used, the rolling and pitching motion time angles are decreased, that is approaching the 0 degree angle described above.

The container includes at least one port to introduce cell cultures, liquid, and other content into the reactor 105 and to remove the introduced cell cultures, liquid, and other content. In another embodiment, the container includes a separate port to function as an outlet port for the introduced cell cultures, liquid, and other content. In one embodiment, the container 105 further includes at least one inlet port which allows for introduction of an appropriately pressurized gas into the container 105 to further give resistance to the smooth circular flow of contents thus increasing proper oxygenation and cell growth. The at least one inlet port can be used to introduce gasses and nutrients to the contents of the container in addition to sampling, harvesting, and placement of sensors.

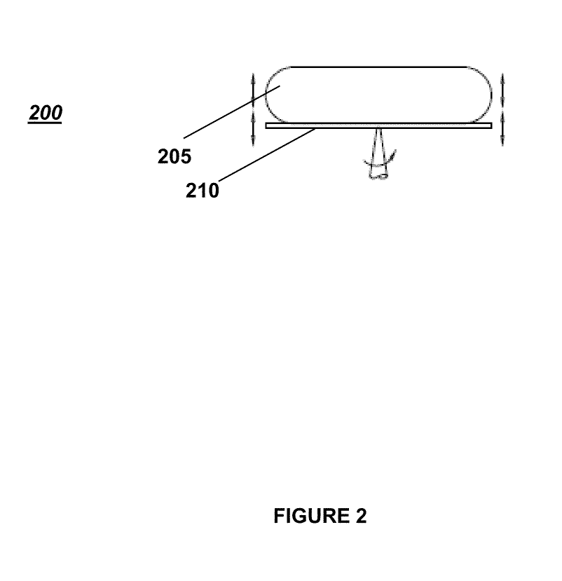

FIG. 2 is a side view of the first embodiment of the continuous flow bioreactor 200 of the present invention as shown in FIG. 1, showing continuous flow bioreactor 205 resting on platform 210. While it should be understood by those of ordinary skill in the art that either a rigid or a disposable configuration may be used, the present invention will now be described with respect to its use as a flexible, disposable bioreactor. In operation of the continuous flow bioreactor of the present invention, clean filtered air is pumped into the flexible bag toroidal container 205, through an input port, to provide the hollow, annular shape and thus, to prevent the walls of the bag from collapsing. The continuous flow bioreactor 205 is placed on top of a container holder (not shown) on platform 210. When actuated, platform 210 has a static, angular 360° swaying motion without actual rotation of the platform, to produce a continuous, circumferential motion to the contents of the reactor 205. In another embodiment, the circumferential motion of the contents of the reactor can be effectuated using bidirectional or multidirectional motion.

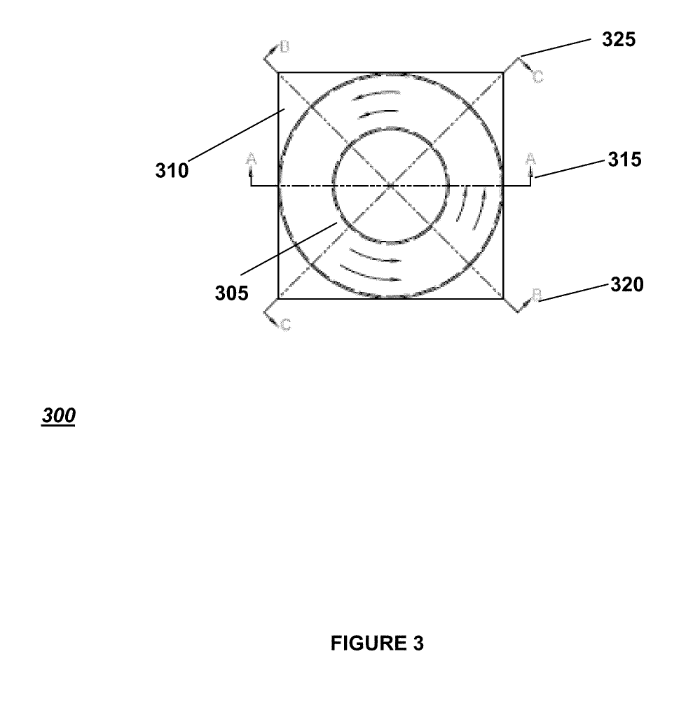

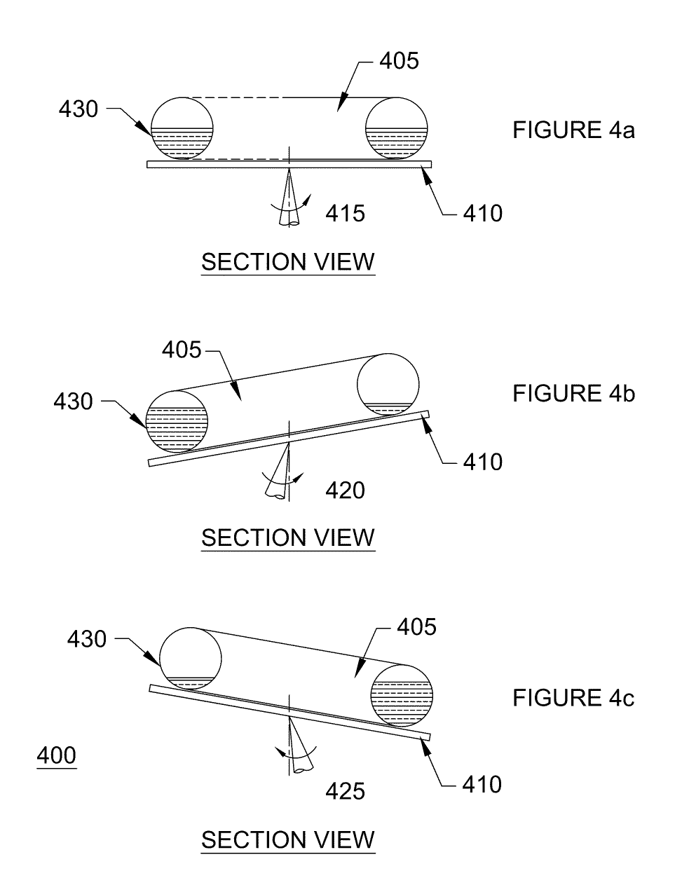

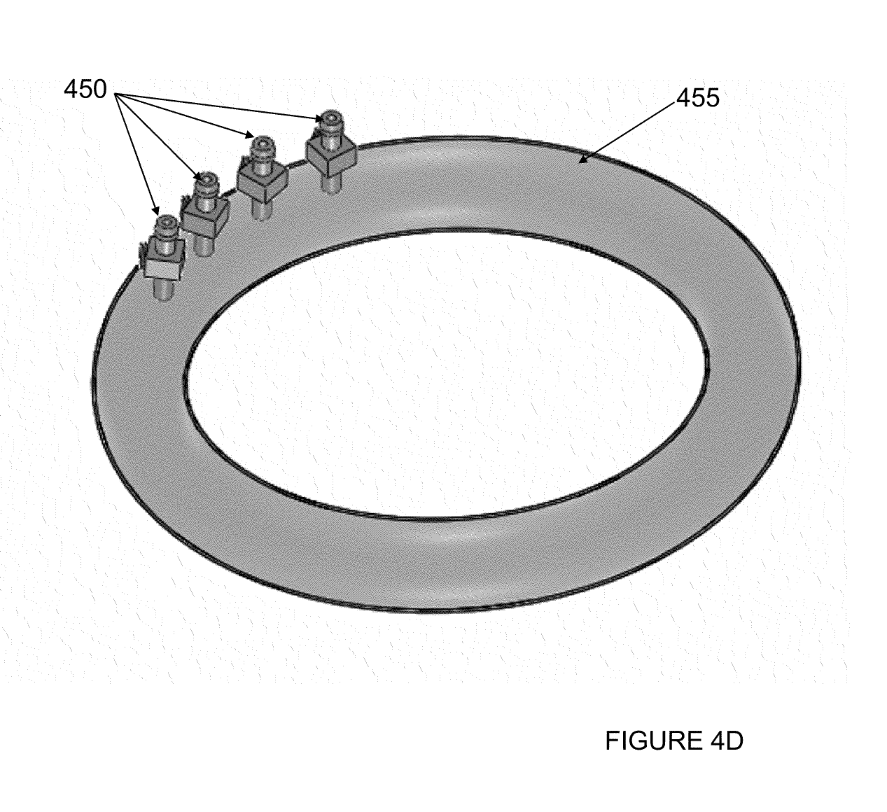

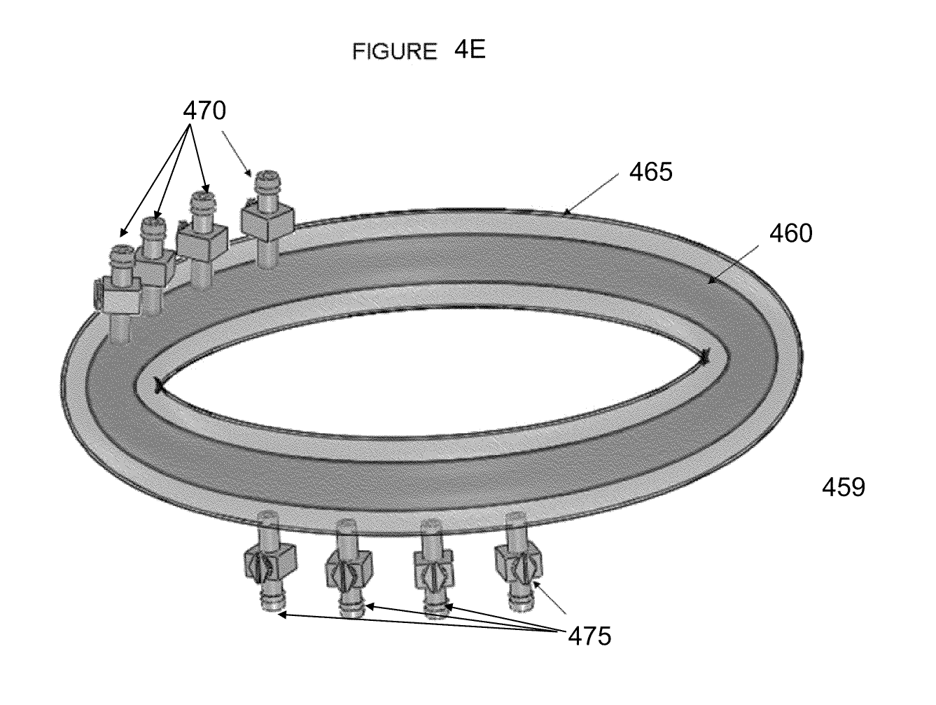

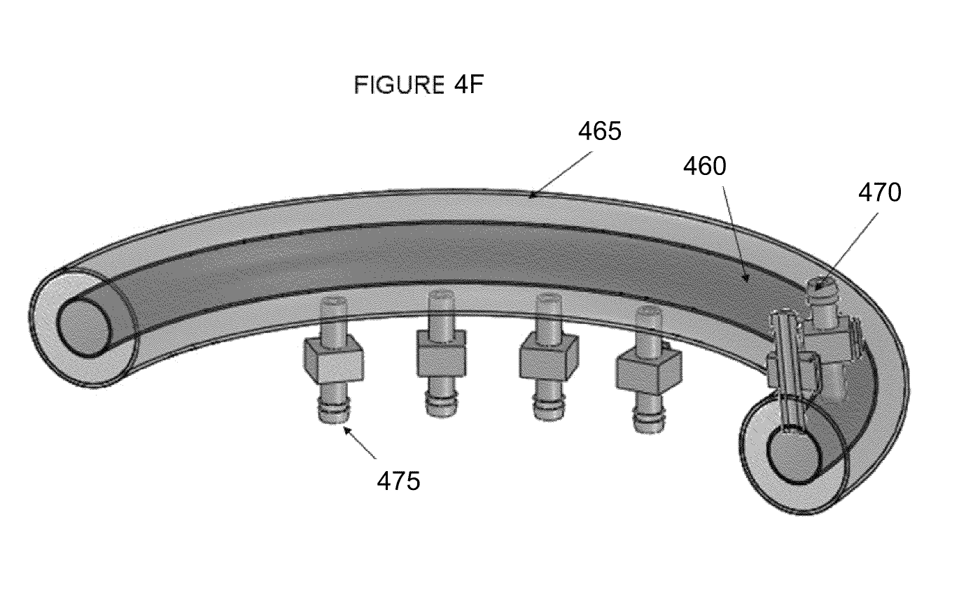

FIG. 3 depicts a top perspective view of the continuous flow bioreactor 305 of the present invention 300, shown in FIG. 1, on platform 310, delineating cross sections 315, 320, and 325, shown in further detail in FIGS. 4a, 4b, and 4c, respectively. FIGS. 4a, 4b, and 4c are sectional side views 415, 420, and 425, respectively of the continuous flow bioreactor 405 of the present invention, in different stages of agitation.

First, FIG. 4a shows the contents of the bioreactor 405 at an equal level when the platform 410 is in horizontal position. However, during angular motion when the platform 410 is tilted to the left, as shown in FIG. 4b, the contents of the bioreactor 405 is at a higher level on the side that is tilted downwards in comparison to the level of content on the opposite side. Next, when angular motion causes the platform 410 to tilt to the right as shown in FIG. 4c, the content of the bioreactor 405 flows circumferentially to accommodate a higher level on the side that is now tilted downwards in comparison to the level of content on the opposite side. Thus, while the content levels 430 at any point of the cross-sectional views of the annular chamber vary from 20% to 80% due to the side-to-side swaying motion, the swaying motion also imparts a gentle sideways movement to the suspended particles in the liquid so as to keep the cells under constant flotation. As the swaying motion progresses from one axis to the other the contents inside the container flows in a continuous circumferential motion and each area of the container will have volume differences and contents in constant motion, thus keeping the cells in a state of suspension and avoiding settling and clumping.

In one embodiment, as shown in FIG. 4d, at least one port or port with an associated filter 450 is optionally placed at the top or bottom of the container 455 to enable sampling, harvesting and/or continuous perfusion of cell cultures.

In one embodiment, as shown in FIGS. 4e and 4f, the perfusion bioreactor 459 of the present invention comprises an annular container housed within an annular container. In one embodiment, inner container 460 comprises a filter membrane that is placed circumferentially along different points of the inner container. Using inner container 460 with a filter membrane at different positions creates a tangential circumferential smooth flow over a large surface area. The contents of the inner container 460 are secreted, at the filter membrane positions, to the outer tube 465, wherefrom secreted products are harvested. In one embodiment, the inner container 460 is a contiguous filter membrane. In another embodiment, the inner container 460 comprises a substantially metal, glass, or plastic housing wall 460 with a filter membrane periodically incorporated therein, placing the interior of the inner container 460 in fluid and/or solid communication with the interior of the outer container 465.

At least one inlet port 470 is located on top of the inner container 460 for feeding nutrients and gasses to the contents of the container 460 and for positioning sensors. At least one outlet port 475 is placed on the bottom side of outer container 465 to harvest secreted product. In addition, due to the large filterable surface area of the inner container 460, and resultant smooth circular flow over the inner filter container, clogging is minimized.

FIG. 5 is a top perspective view of a second embodiment of the continuous flow bioreactor of the present invention. In the second embodiment, the continuous flow bioreactor 500 is agitated by an agitation means connected to the bottom surface of platform 510. The smooth, circular motion of the contents is created by a triple axial motion of pitch, yaw, and roll, which is effectuated by programmable motion of the motors. Because of the arrangement of the platform and agitation means, the embodiment shown in FIG. 5 can accommodate up to 1000 liters depending on the diameter and circumference of the container. For example, a container having a diameter of 1 foot and a circumference of 5 feet, in one embodiment, accommodates 330 liters.

As shown in FIG. 5, bioreactor 500 comprises container/tube 505 which rests on platform 510, using expandable container holder 511. As described with respect to FIG. 1 above, container 505 is in the shape of a toroid and, in one embodiment the walls of container 505 form a central opening 506 so that the container 505 is an annular chamber capable of housing contents such as cell cultures. In one embodiment, container 505 resembles the shape of a standard bicycle tube.

In one embodiment, the container 505 is rigid and formed from an appropriate firm material such as stainless steel or glass. In another embodiment, the container 505 is disposable and is formed from an appropriate material such as flexible plastic (disposable bag) that is inflatable using a filtered air stream and can be used for a variety of cell cultures. The flexible container can be pre-sterilized and can be disposable

Platform 510 may be of any shape, but is preferably square, circular or rectangular. Further, platform 510 is movably attached to a support 515 at its proximal end 515a. Platform 510 is connected to support base platform 582 of agitation mechanism at its distal end 515b. Support 515 is, in one embodiment, used to provide a smooth movement means for platform 510 such that an agitation mechanism can be used to easily move the platform.

The agitation mechanism comprises a base support platform 582 for connecting a plurality of components, including programmable motors 584, 586, and 588, which control movement along three axes (roll, pitch, and yaw). Programmable motors 584, 586, and 588 are each equipped with levers 583, 585, and 587, respectively for moving platform 510 along its three axes. Members, rods, or levers 583, 585, and 587 are connected to motors 584, 586, and 588, respectively, at their distal ends 583a, 585a, and 587a, respectively and to platform 510 at their proximal ends 583b, 585b, and 587b, respectively. It should be appreciated that the agitation mechanism may be controlled in the form and method as described above with respect to the first embodiment.

When actuated, platform 510 has a static, angular 360° swaying motion (similar to combined rolling and pitching action and described in detail below) with integrated swaying of the platform and effectuates a continuous, circumferential motion to the contents of the reactor 500. In another embodiment, the circumferential motion of the contents of the reactor can be effectuated using bidirectional or multidirectional motion. The motion of the platform 510 and hence the resulting circumferential motion of the contents of container 505, therefrom, is understood with reference to a set of axes—a) vertical axis ‘V’ 550 that passes through the center of the platform 510 and along which also resides the connector member 515 in a parallel orientation; b) longitudinal axis ‘L’ 555 and c) transverse axis ‘T’ 560.

In one embodiment, the agitation mechanism effectuates sequential motions of the platform 510 and the container 505, as shown in FIGS. 5a, 5b, 5c, and 5d, which are top perspective views of the second embodiment of the continuous flow bioreactor 500 of the present invention, shown in FIG. 5, in different stages of agitation. Thus, in one embodiment, the agitation mechanism effectuates repeated sequential motions of the platform 510 and the reactor 505: a first rolling motion 525 around the longitudinal axis ‘L’ 555 and a second pitching motion 520 around the transverse axis ‘T’ 560 along with a simultaneous rotational motion around the vertical axis ‘V’ 550. For example, the platform 510 may first have angular motion 520 followed by the angular motion 525, or vice versa, repeated one after another in a continuous closed loop for a specified duration of time. In addition a rotational motion 530 simultaneously causes a 360 degree motion of the contents of reactor container 505.

As shown in FIG. 5A, the agitation mechanism 580 causes the fluid 590 within container 505 to tilt towards area 590B on side defined by line A-B and away from area 590A defined by line C-D. Fluid motion is effectuated by a rotation of axis L 555 about vertical axis V 550 in the direction of line A-B. This motion is achieved by the programmed movement of members, rods, or levers 583, 585, and 587 by 584, 586, and 588, and, in particular, the upward movement of lever 585 by motor 586 and downward movement of level 583 by motor 584. As would be appreciated by one of ordinary skill in the art, the movement of the motors is effectuated by controllers as driven by user input instructions.

As shown in FIG. 5B, the agitation mechanism 580 causes the fluid 590 within container 505 to tilt towards corner 593 (B). Fluid motion is effectuated by a rotation of axis L 555 about vertical axis V 550 in the direction of corner 593 (B). Fluid lines 590a and 590b show the end points of the volume of fluid, when the platform is moved toward corner 593 (B). This motion is achieved by the programmed movement of members, rods, or levers 583, 585, and 587 by 584, 586, and 588, and, in particular, the upward movement of lever 585 by motor 586 and/or upward movement of level 587 by motor 588 and the stationary or downward movement of level 583 by motor 584. As would be appreciated by one of ordinary skill in the art, the movement of the motors is effectuated by controllers as driven by user input instructions.

As shown in FIG. 5C, the agitation mechanism 580 causes the fluid 590 within container 505 to tilt towards corner 595 (C). Fluid motion is effectuated by a rotation of axis L 555 about vertical axis V 550 in the direction of corner 595 (C). Fluid lines 590a and 590b show the end points of the volume of fluid, when the platform is moved toward corner 595 (C). This motion is achieved by the programmed movement of members, rods, or levers 583, 585, and 587 by 584, 586, and 588, and, in particular, the upward movement of lever 587 by motor 588 and the stationary or downward movement of level 585 by motor 586. As would be appreciated by one of ordinary skill in the art, the movement of the motors is effectuated by controllers as driven by user input instructions.

As shown in FIG. 5D, the agitation mechanism 580 causes the fluid 590 within container 505 to tilt towards corner 597 (D). Fluid motion is effectuated by a rotation of axis L 555 about vertical axis V 550 in the direction of corner 597 (D). Fluid lines 590a and 590b show the end points of the volume of fluid, when the platform is moved toward corner 597 (D). This motion is achieved by the programmed movement of members, rods, or levers 583, 585, and 587 by 584, 586, and 588, and, in particular, the upward movement of lever 583 by motor 584 and the stationary or downward movement of level 585 by motor 586. As would be appreciated by one of ordinary skill in the art, the movement of the motors is effectuated by controllers as driven by user input instructions.

It should be appreciated that the each motor causes a translational movement, either upward or downward, in a member attached to the motor, which, in turn, causes a corresponding translational movement in the area of the platform (and thus the area of the container proximate to that area of the platform) to which the member is attached. Any sequence of translational movement can be effectuated by the controller. It should further be appreciated that each sequence of up or down movement can be separated by a rest period, in which all areas of the platform return to a flat, initial position, or can be performed continuously with one translational movement (i.e. upward movement of lever 585 and the area of the platform, and therefore the portion of the container proximate to such area, to which lever 585 is attached) followed immediately, followed substantially concurrently, or overlapped with a second translational movement (i.e. upward movement of lever 583 and the area of the platform, and therefore the portion of the container proximate to such area, to which lever 583 is attached) and/or with a third translational movement (i.e. downward movement of lever 587 and the area of the platform, and therefore the portion of the container proximate to such area, to which lever 587 is attached). It should further be appreciated that additional motors and levers can be added to cause translational movement to a fourth, fifth, sixth, seventh, eighth, ninth, tenth, or more area of the platform. It also be appreciated that, while a single motor has been depicted as being the cause of the translational movement of a single lever, one motor can be used to cause an independent translational movement to two or more different levers.

Referring back to FIG. 5, a spherical knob at proximal end 515a enables smoother motion of the platform 515 and hence of the reactor 510 and its contents. The continuous and repeated combination of the two angular motions 520, 525 combined with the rotational motion 530 results into a continuous, side-to-side as well as circumferential motion of the contents of the reactor 505, around vertical axis ‘V’ 550. Thus, the contents of the reactor 505 are agitated with a combination of side-to-side swaying against walls of the reactor 505 as well as a circumferential motion 530 around vertical axis ‘V’ 550. In one embodiment, the speed of the motion depends upon at least one of the working load and the density and viscosity of the medium/media present in the bioreactor.

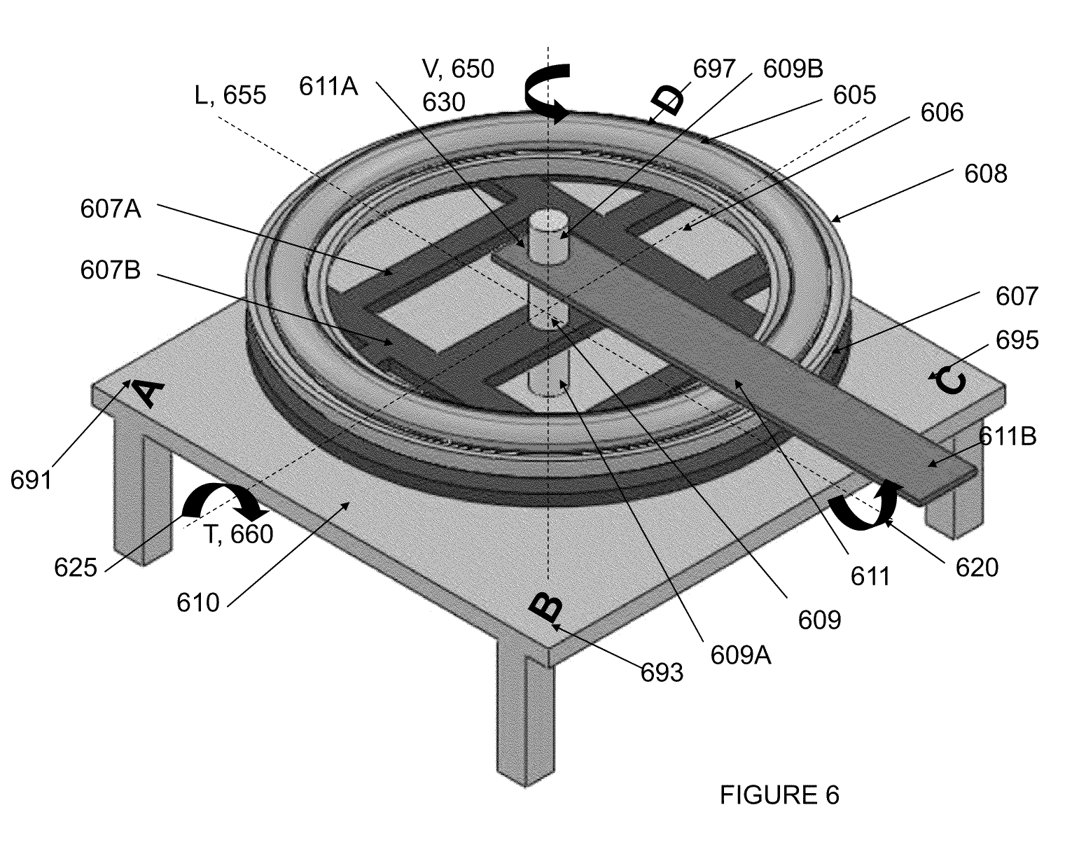

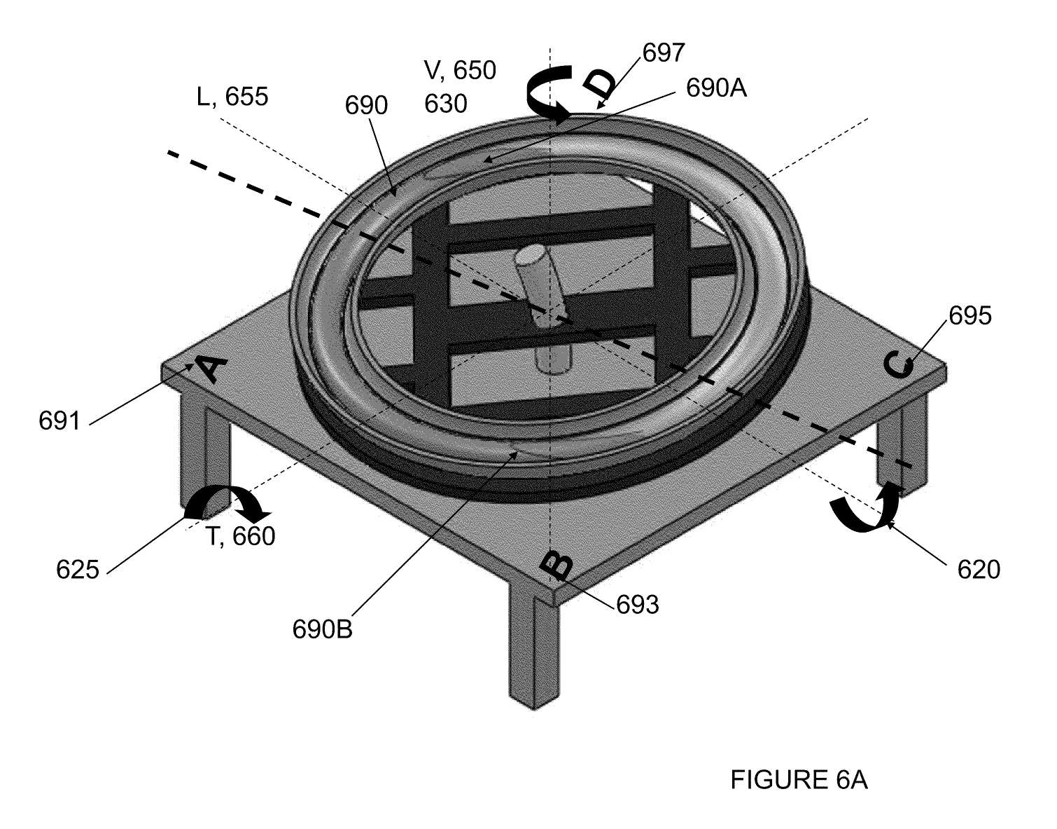

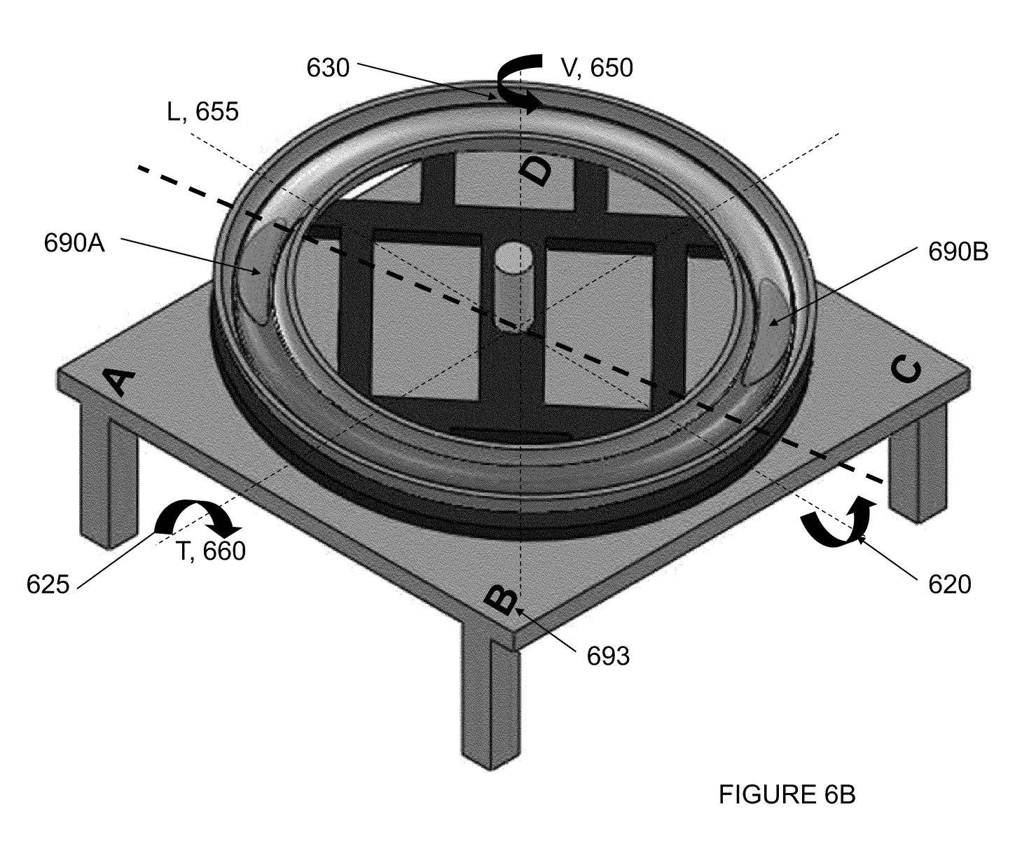

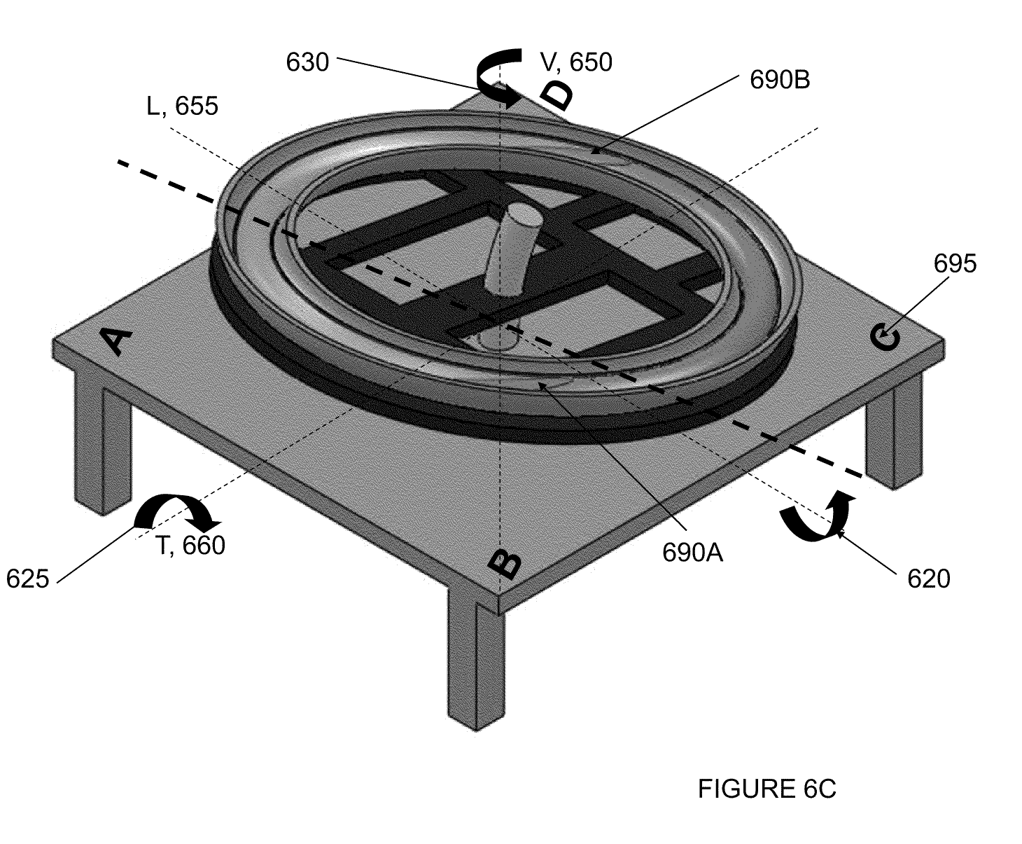

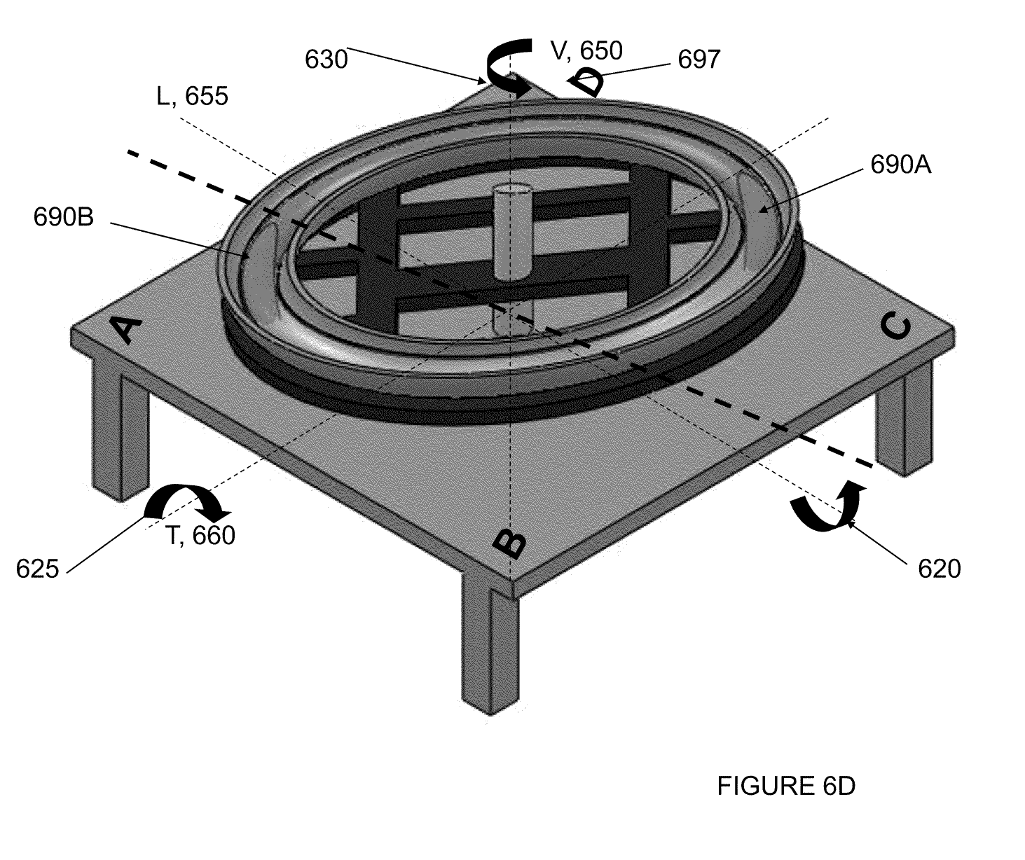

FIG. 6 is a top perspective view of a third embodiment of the continuous flow bioreactor of the present invention. In the third embodiment, the continuous flow bioreactor of the present invention has a self-contained agitation mechanism. Thus, as shown in FIG. 6, continuous flow bioreactor 600 comprises a container 605, platform 610, and expandable container holder 608. As described with respect to FIGS. 1 and 5 above, container 605 is in the shape of a toroid and in one embodiment the walls of container 605 form a central opening 606 so that the container 605 is an annular chamber capable of housing contents such as cell cultures. In one embodiment, container 605 resembles the shape of a standard bicycle tube.

In one embodiment, the container 605 is rigid and formed from an appropriate material such as stainless steel or glass. In another embodiment, the container 605 is disposable and is formed from an appropriate material such as a flexible plastic (disposable bag) that is inflatable using a filtered air stream and can be used for a variety of cell cultures.

The continuous flow bioreactor 600 of the present invention further comprises a rim 607, which in one embodiment, further comprises a central hub 609 and a plurality of transverse spokes or supports 607a and a plurality of longitudinal spokes or supports 607b. In one embodiment, expandable container holder 608 is attached to a top surface of the rim 607, as shown in FIG. 6. The lower end 609a of the central hub sits in a plurality of bearings and is movably attached to a central point on platform 610 such that it has free motion in the three axial directions. Hub 609 is also employed to elevate the rim 607 and container 605 a suitable minimum distance ranging from 6 to 12 inches from the platform 610. In one embodiment, hub 609 acts as a self-contained agitation mechanism, as only the hub 609 needs to be swayed to effectuate movement of the continuous flow bioreactor 600.

In one embodiment, the top end 609b of the hub 609 is connected to the proximal end 611a of a lever 611. In one embodiment, at its distal end 611b, lever 611 is connected to a programmable motor to create smooth gyration of the rim 607, causing at least one point on the rim 607 to be at a titled angle position at all times. In one embodiment, the point on rim 607 at a tilted angle is shifted constantly to create a smooth circular motion of the fluid. In other embodiments, the external agitation mechanism is employed to move the hub in three axial directions and is actuated by mechanical means, electrical means, and can be fixed at top end of the hub or at the bottom end of the hub.

Platform 610 may be of any shape, but is preferably circular or rectangular. When moved, rim 607 (moved using hub 609), and thus container 605, has a static, angular 360° swaying motion (similar to combined rolling and pitching action) to produce a continuous, circumferential motion to the contents of the reactor 605. In another embodiment, the circumferential motion of the contents of the reactor can be effectuated using bidirectional or multidirectional motion.

FIGS. 6a, 6b, 6c, and 6d are top perspective views of a third embodiment of the continuous flow bioreactor of the present invention, in different stages of agitation.

As shown in FIG. 6a, using hub 609, container 605 is moved along longitudinal axis 655 (L) about vertical axis 650 (V) causing the fluid 690 within container 605 to tilt towards corner 691 (A). Fluid lines 690a and 690b show the end points of the volume of the fluid as moved by the tilting container. This motion is achieved by the programmed movement of member 611 by application of pressure at distal end 611B by a motor in the direction of corner 691 (A).

As shown in FIG. 6b, using hub 609, container 605 is moved along longitudinal axis 655 (L) about vertical axis 650 (V) causing the fluid 690 within container 605 to tilt towards corner 693 (B). Fluid lines 690a and 690b show the end points of the volume of the fluid as moved by the tilting container. This motion is achieved by the programmed movement of member 611 by application of pressure at distal end 611B by a motor in the direction of corner 693 (B).

As shown in FIG. 6c, using hub 609, container 605 is moved along longitudinal axis 655 (L) about vertical axis 650 (V) causing the fluid 690 within container 605 to tilt towards corner 695 (C). Fluid lines 690a and 690b show the end points of the volume of the fluid as moved by the tilting container. This motion is achieved by the programmed movement of member 611 by application of pressure at distal end 611B by a motor in the direction of corner 695 (C).

As shown in FIG. 6d, using hub 609, container 605 is moved along longitudinal axis 655 (L) about vertical axis 650 (V) causing the fluid 690 within container 605 to tilt towards corner 697 (D). Fluid lines 690a and 690b show the end points of the volume of the fluid as moved by the tilting container. This motion is achieved by the programmed movement of member 611 by application of pressure at distal end 611B by a motor in the direction of corner 697 (D).

In one embodiment, as shown in FIGS. 4e and 4f, at least one top port and one bottom port can be positioned on the container for adding nutrients, gasses and sensors and for placing filters for harvesting or perfusion. Thus, referring back to FIG. 6, in one embodiment, the container 605 further includes an inlet port which allows for introduction of an appropriately pressurized gas into the container 605 to lend further resistance to the contents of the container. The inlet port is gas leak proof and, in one embodiment, is designed for use with an inert gas.

In another embodiment, a perfusion bioreactor is created utilizing a “tube within a tube” design. In one embodiment, both tubes can be made of flexible or rigid materials, can be pre-sterilized, and can be disposable. In one embodiment, the inner tube comprises a filter membrane, entirely, where cellular, viral, bacterial, plant or animal cell activity takes place. The secreted products are filtered out due to tangential flow of the fluid in the inner tube. The products are filtered in to the outer tube.

In one embodiment, the inner tube can have multiple ports or inlets on the top surface for introduction of nutrients and/or gasses and for placement of sensors. In one embodiment, the outer tube can have multiple ports or inlets on the bottom surface for harvesting the product filtered out of the inner tube. In another embodiment, a slight negative pressure can be applied to the outer tube via the available ports to facilitate better filtration.

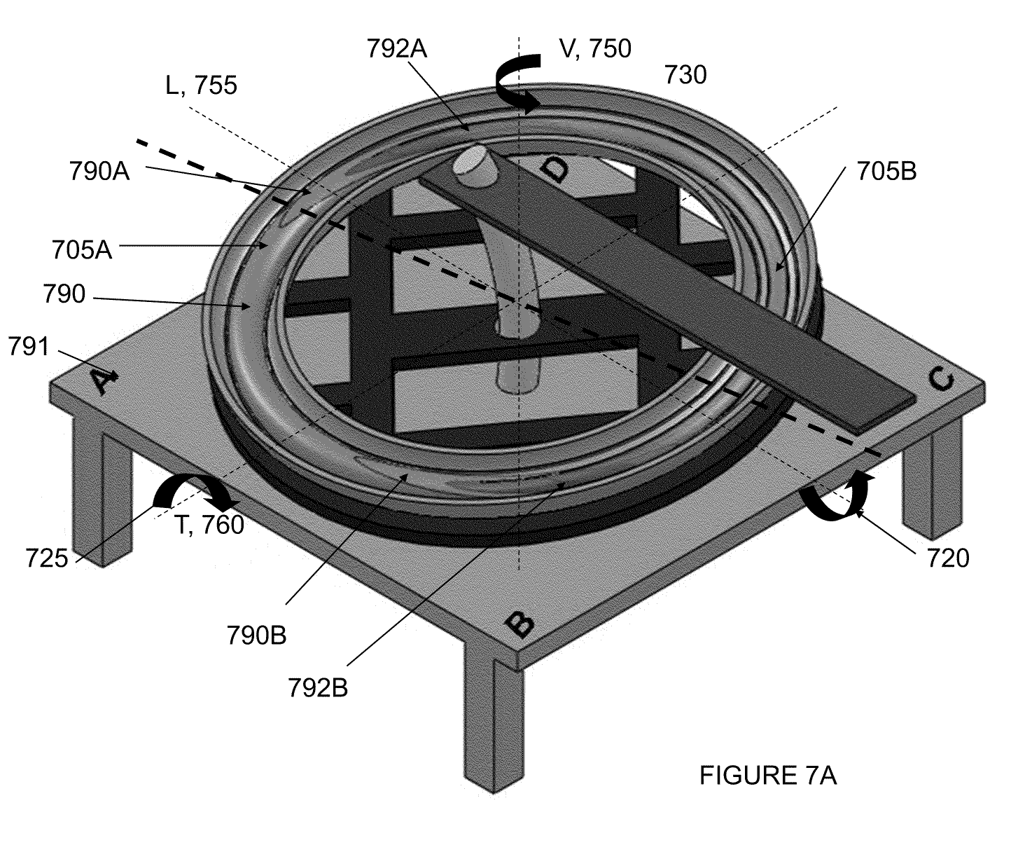

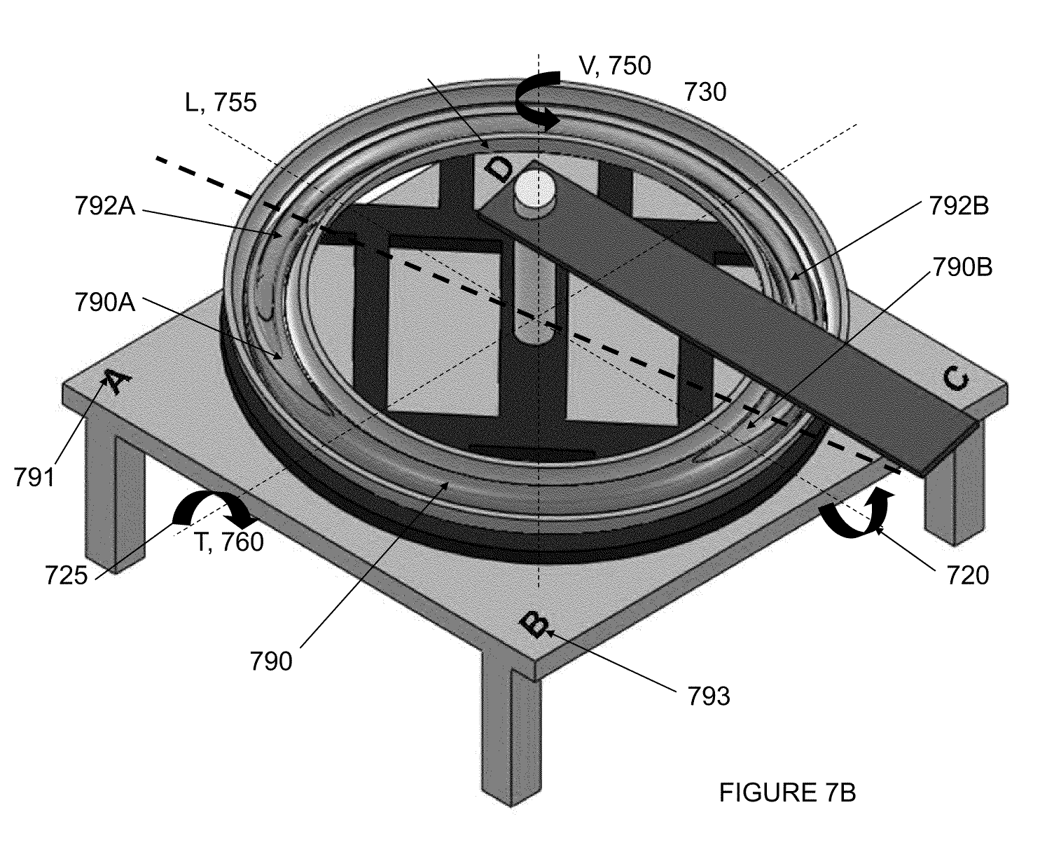

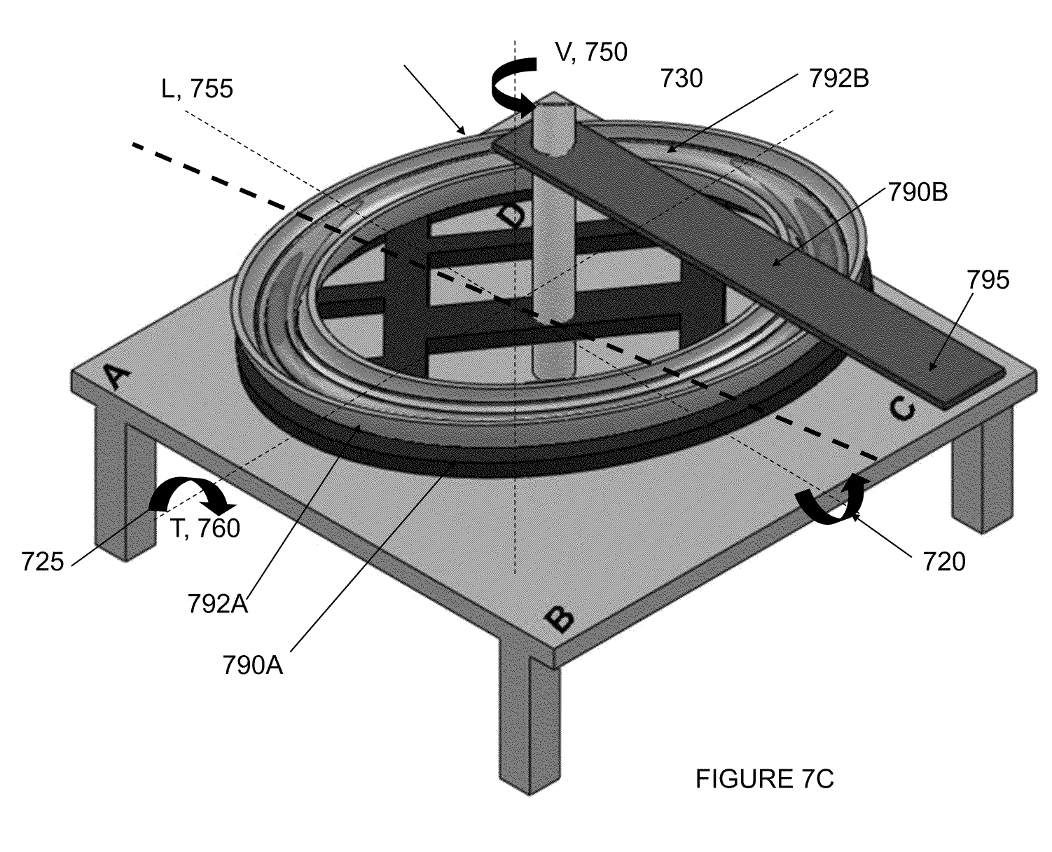

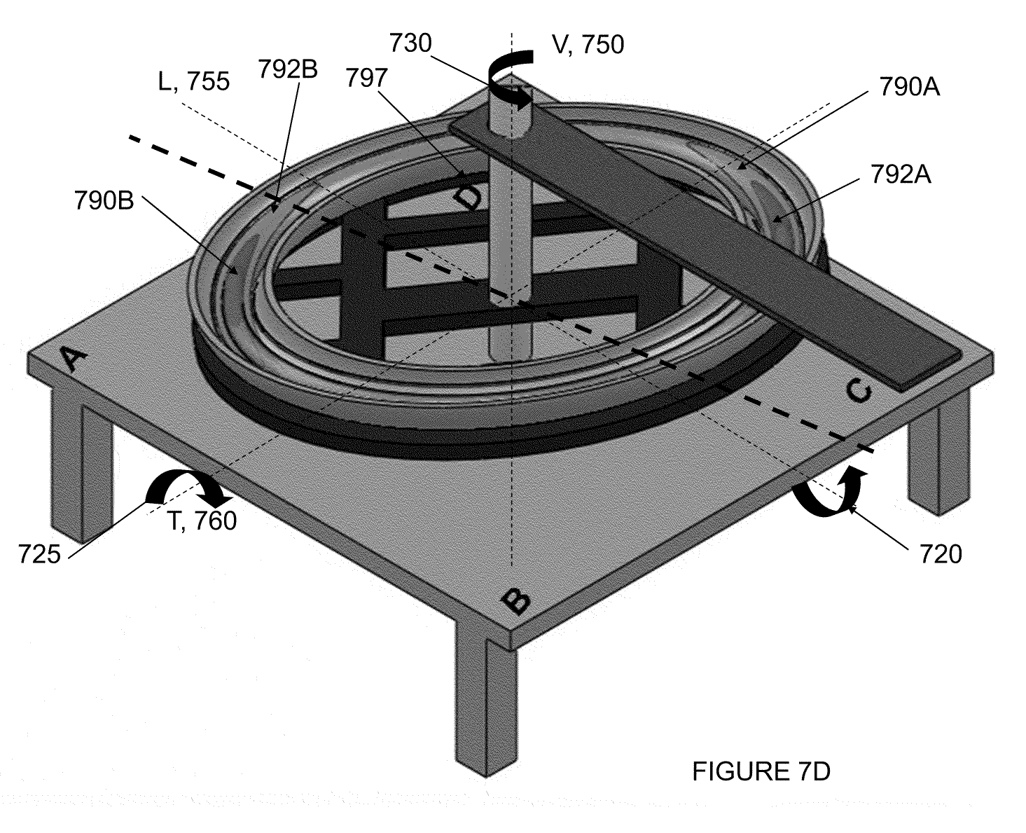

FIGS. 7a, 7b, 7c, and 7d are illustrations of the perfusion bioreactor of the present invention at various stages of agitation. Shown in FIG. 7a is an embodiment similar to FIG. 6, except the container has outer tube 705a and inner tube 705b. When the bioreactor is tilted, as described with respect to FIG. 6a above, fluid 790 within outer tube 705a and inner tube 705b tilts towards corner 791 (A). Fluid lines 790a and 790b show the end points of the volume of the fluid within outer tube 705a and fluid lines 792a and 792b show the end points of the volume of the fluid within inner tube 705b.

FIG. 7b shows container 705 having outer tube 705a and inner tube 705b. When the bioreactor is tilted, as described with respect to FIG. 6b above, fluid 790 within outer tube 705a and inner tube 705b tilts towards corner 793 (B). Fluid lines 790a and 790b show the end points of the volume of fluid within the outer tube and fluid lines 792a and 792b show the end points of the volume of fluid within inner tube 705b.

FIG. 7c shows container 705 having outer tube 705a and inner tube 705b. When the bioreactor is tilted, as described with respect to FIG. 6c above, fluid 790 within outer tube 705a and inner tube 705b tilts towards corner 795 (C). Fluid lines 790a and 790b show the end points of the volume of fluid within outer tube 705a and fluid lines 792a and 792b show the end points of the volume of fluid within inner tube 705b.

FIG. 7d shows container 705 having outer tube 705a and inner tube 705b. When the bioreactor is tilted, as described with respect to FIG. 6d above, fluid 790 within outer tube 705a and inner tube 705b tilts towards corner 797 (D). Fluid lines 790a and 790b show the end points of the volume of fluid within outer tube 705a and fluid lines 792a and 792b show the end points of the volume of fluid within inner tube 705b.

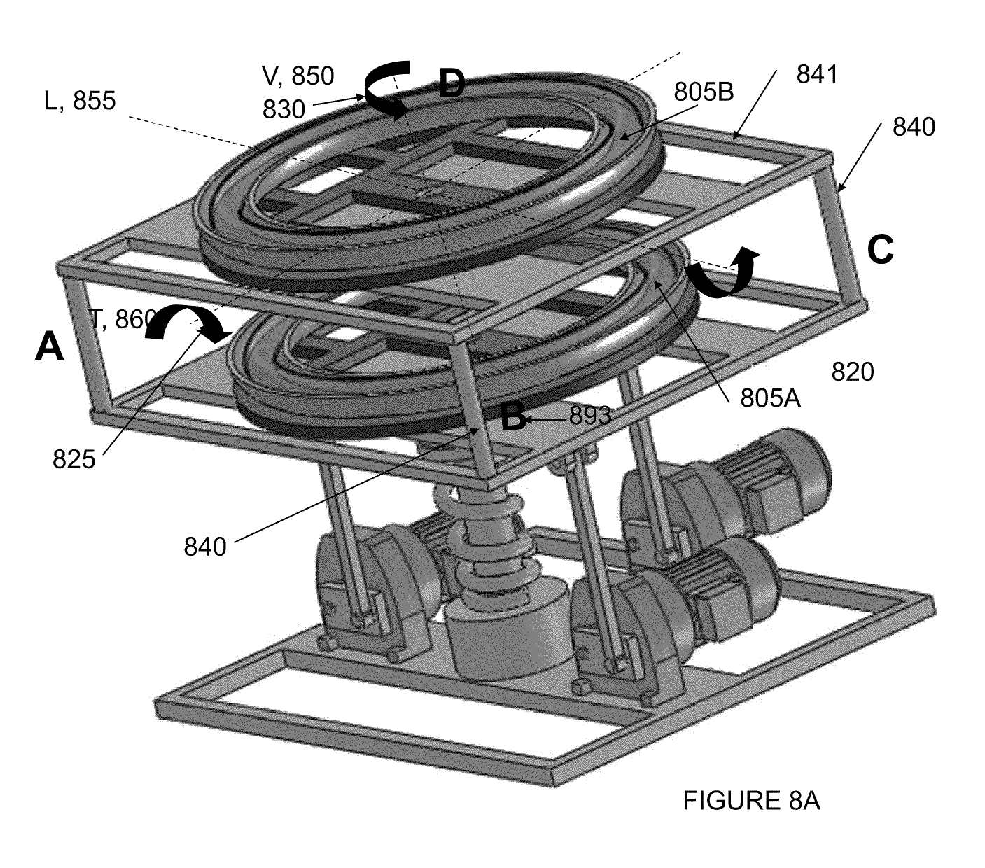

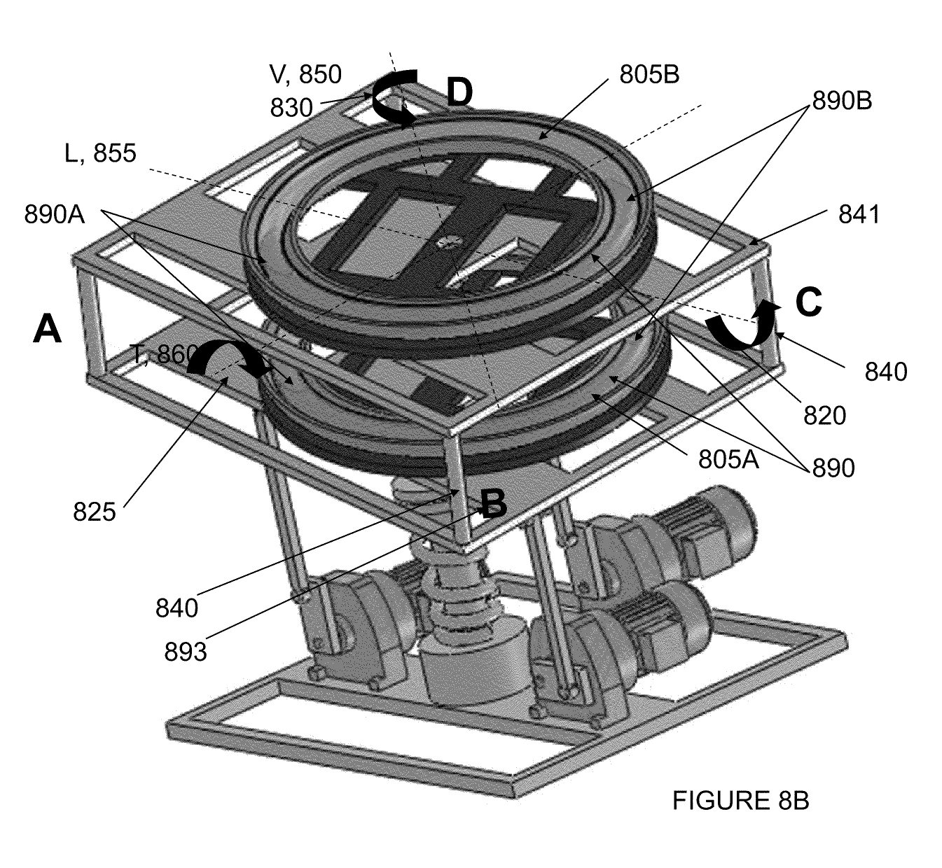

In another embodiment, as shown in FIG. 8A, the bioreactor can be used in a stacked configuration. Thus, in this configuration, a top bioreactor container 805b can be placed over a bottom bioreactor container 805a, on the same support platform, that comprises an additional base for supporting the top bioreactor container 805b. The base components of the bioreactor have been described in great detail with respect to FIGS. 5, 5a, 5b, 5c, and 5d and will not be described in detail herein. The embodiments shown in FIGS. 8a and 8b will only be described with respect to the differences contained therein.

In one embodiment, the top bioreactor container 805b is positioned on top of upper support platform 841, which is connected to lower support platform 842 using four removable connectors 840 at each of four corners, and a removable connector in the center, that translates motion in the lower connector rod to motion in the upper connector rod. The triple axis motion (pitch, roll, and yaw), as described in FIGS. 5a, 5b, 5c, and 5d is transmitted to the upper levels of the bioreactor. It should be noted herein that a plurality of stacked levels may be used in the same manner, depending upon height space availability and platform capacity.

FIG. 8a is a depiction of a stackable bioreactor 800, in one embodiment of the present invention, having two bioreactors and thus, two levels, in a neutral position. Stackable bioreactor 800 moves along the longitudinal axis 855 about the vertical axis 850, towards corner A, such that fluid 890 contained within the containers 805a and 805b sways toward corner A.

As shown in FIG. 8b, stackable bioreactor 800 and thus, containers 805a and 805b, are moved along longitudinal axis 855 (L) about vertical axis 850 (V) causing the fluid 890 within the containers 805a and 805b to tilt towards corner 893 (B). Fluid lines 890a and 890b show the end points of the volume of fluid, in this embodiment.

In another embodiment, the hub and rim design described with respect to FIGS. 6, 6a, 6b, 6c, and 6d can also have a stackable configuration. Thus, the total volume capacity of the bioreactor can be increased to greater than 1000 liters. In this embodiment, the stacking can be anywhere from a one level system to a n-level system, depending upon height and load factors. For example, in one embodiment, it is possible to stack three levels of a 1 foot diameter, 5 foot circumference container each having a volume capacity of 330 liters. This configuration is advantageous because each stacked container can operate as an independent unit, thus decreasing the overall load.

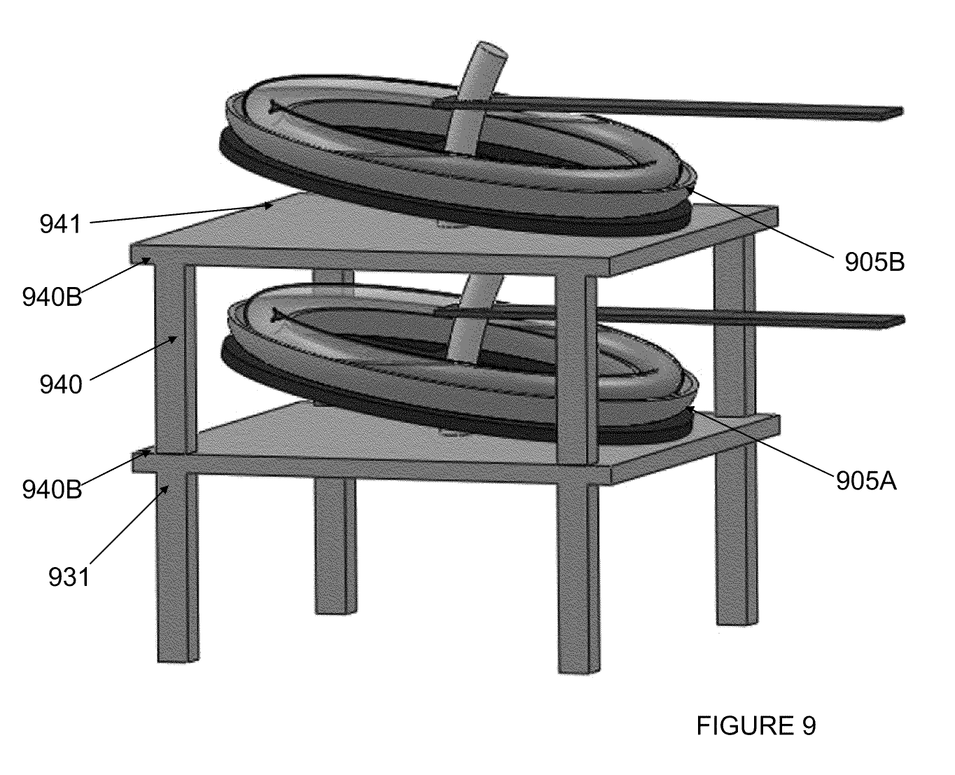

FIG. 9 is a depiction of the hub and rim design described with respect to FIG. 6 in a stacked configuration. FIG. 9a shows lower bioreactor container 905a and upper bioreactor container 905b in a stacked configuration. To achieve the stacking, an upper platform 941 is connected to a lower platform 931 by use of four removable connecting supporting rods 940 connected to the lower platform 931 at their lower end 940a and to the upper platform 941 at their upper end 940b. Both bioreactors can be agitated by linking their actuating levers to separate motors, which has already been described with respect to FIG. 6 and will not be repeated herein.

While the exemplary embodiments of the present invention are described and illustrated herein, it will be appreciated that they are merely illustrative. It will be understood by those skilled in the art that various changes in form and detail may be made therein without departing from or offending the spirit and scope of the appended claims.

We claim:

1. A bioreactor system comprising:
a. A base platform having a flat surface, a first area, a second area, and a third area, wherein each of said first, second, and third areas are separated by a distance;
b. A toroidal container removably attached to said base platform;
c. A first member physically attached to said first area;
d. A second member physically attached to said second area;
e. A third member physically attached to said third area; and
f. A plurality of motors, wherein each of said motors is configured to cause an independent translational movement in said first member, said second member, and said third member, and wherein said translation movement in each of said first member, second member, and third member causes a corresponding translational movement in a portion of said toroidal container proximate to said first area, said second area, and said third area, respectively.

2. The bioreactor system of claim 1 wherein said translational movement is either upward or downward.

3. The bioreactor system of claim 1 further comprising a controller for controlling a sequence of said translational movement.

4. The bioreactor system of claim 3 wherein said sequence comprises causing an upward translational movement of said first area substantially concurrently with causing a downward translational movement of said second area.

5. The bioreactor system of claim 4 wherein said sequence further comprises, after said causing an upward translational movement of said first area substantially concurrently with causing a downward translational movement of said second area, causing an upward translational movement of said second area substantially concurrently with causing a downward translational movement of said first area.

6. The bioreactor system of claim 5 wherein said sequence causes contents within said toroidal container to have an angular motion throughout said toroidal container without requiring a physical rotation of said container.

7. The bioreactor system of claim 1 wherein said plurality of motors comprises a first motor, a second motor and a third motor, wherein said first motor is configured to cause a translational movement in said first member, said second motor is configured to cause a translational movement in said second member, and said third motor is configured to cause a translational movement is said third member.

8. The bioreactor system of claim 1 wherein said platform rests atop a support member comprising a pivot point.

9. The bioreactor system of claim 8 wherein said pivot point comprises a spherical surface to which said a bottom surface of said platform is attached.

10. The bioreactor system of claim 1 wherein said toroidal container has a first internal volume and a second internal volume, wherein said second internal volume is defined by a second toroidal container housed within said toroidal container.

11. The bioreactor system of claim 10 wherein said second toroidal container comprises a filter.

12. The bioreactor system of claim 10 wherein said second toroidal container comprises an input port.

13. The bioreactor system of claim 10 wherein said toroidal container comprises an output port.

14. A bioreactor system comprising:
a. A base platform having a flat surface, a first area, a second area, and a third area, wherein each of said first, second, and third areas are separated by a distance;
b. A container removably attached to said base platform;
c. A first member physically attached to said first area;
d. A second member physically attached to said second area;
e. A third member physically attached to said third area;
f. A first motor, wherein said first motor is configured to cause a first translational movement in said first member;
g. A second motor, wherein said second motor is configured to cause a second translational movement in said second member; and
h. A third motor, wherein said third motor is configured to cause a third translational movement in said third member, wherein each of said first, second, and third translational movements are capable of occurring independent of each other and wherein said first, second, and third translation movements by each of said first, second, and third members causes a corresponding translational movement in a portion of said toroidal container proximate to said first area, said second area, and said third area, respectively.

15. The bioreactor system of claim 14 further comprising a controller for controlling a sequence of said translational movement.

16. The bioreactor system of claim 15 wherein said sequence comprises causing an upward translational movement of said first area substantially concurrently with causing a downward translational movement of said second area.

17. The bioreactor system of claim 16 wherein said sequence further comprises, after said causing an upward translational movement of said first area substantially concurrently with causing a downward translational movement of said second area, causing an upward translational movement of said second area substantially concurrently with causing a downward translational movement of said first area.

18. The bioreactor system of claim 17 wherein said container is toroidal and wherein said sequence causes contents within said toroidal container to have an angular motion throughout said toroidal container without requiring a physical rotation of said container.

19. The bioreactor system of claim 14 wherein said container is toroidal and wherein said toroidal container has a first internal volume and a second internal volume, wherein said second internal volume is defined by a second toroidal container housed within said toroidal container.

20. The bioreactor system of claim 19 wherein said second toroidal container comprises a filter.
